(19) United States
(12) Patent Application Publication
(10) Pub. No.: US 2021/0144999 A1
(43) Pub. Date: May 20, 2021

(54) COMPOSITIONS COMPRISING HYDROXYCINNAMIC ACID AND RELATED CONJUGATES, ANALOGUES AND DERIVATIVES, AND METHODS OF USE THEREOF

(71) Applicants: YEDA RESEARCH AND DEVELOPMENT CO. LTD., Rehovot (IL); THE NATIONAL INSTITUTE FOR BIOTECHNOLOGY IN THE NEGEV LTD., Beer Sheva (IL)

(72) Inventors: Ilana KOLODKIN-GAL, Rehovot (IL); Yaara OPPENHEIMER-SHAANAN, Rehovot (IL); Ariel OGRAN-NACHMAN, Rehovot (IL); Michael MEIJLER, Omer (IL); Eliane Hadas YARDENI, Rehovot (IL)

(73) Assignees: YEDA RESEARCH AND DEVELOPMENT CO. LTD., Rehovot (IL); THE NATIONAL INSTITUTE FOR BIOTECHNOLOGY IN THE NEGEV LTD., Beer Sheva (IL)

(21) Appl. No.: 16/629,572

(22) PCT Filed: Jul. 9, 2018
(86) PCT No.: PCT/IL2018/050744

§ 371 Date: Jan. 9, 2020

(30) Foreign Application Priority Data
Jul. 9, 2017 (IL) 253370

(51) Int. Cl. A01N 43/16; A01N 37/38; A01N 33/04; C07C 231/24; C12N 1/38

(52) U.S. Cl. CPC A01N 43/16; A01N 37/38; C12N 1/38; C07C 231/24; A01N 33/04

(57) ABSTRACT

The present invention provides anti-microbial compositions comprising a hydroxycinnamic acid and related conjugates, analogues and derivatives, and methods of use thereof for enhancing production of anti-microbial agents in bacteria as well as for protecting plants from microbial infection.

### FIELD OF INVENTION

The present invention provides anti-microbial compositions comprising a hydroxycinnamic acid and related conjugates, analogues and derivatives, and methods of use thereof for enhancing production of anti-microbial agents in bacteria as well as for protecting plants from microbial infection.

### BACKGROUND OF THE INVENTION

Bacillus subtilis is a bacterial species that is highly relevant in agriculture and is positioned to dominate the bio-control field. Naturally present in the immediate vicinity of plant roots, B. subtilis is able to maintain stable contact with higher plants and promote their growth. In addition, Bacillus subtilis has a broad host range [including tobacco, tomato, melons and many more commercial crops], and is completely benign to humans, and does not survive in a human host.

Importantly, B. subtilis and related species defend plants from diverse fungal and bacterial pathogens. This protection is mediated, in part, by the formation of biofilms, which are a differentiated complex community of bacterial cells held together by an extracellular matrix.

Moreover, B. subtilis can produce a great abundance of antibiotics with an amazing variety of structures and activities and can suppress more than 23 types of plant pathogens in vitro. Antibiotics produced by Bacillus subtilis are resistant to hydrolysis by peptidases and proteases and to changes in temperature and pH. For these reasons, B. subtilis as well as other Bacilli are potentially leading candidates as bio-control agents.

Understanding how biofilm formation protects plants from pathogens may lead to new and safer methods of crop protection.

### SUMMARY OF THE INVENTION

In one embodiment, the present invention provides a method of synthesizing an anti-microbial agent in bacteria, enhancing the production of an anti-microbial agent in bacteria, activating the pks gene operon in bacteria having a pks gene operon, or a combination thereof, the method comprising administering to said bacteria a composition comprising a hydroxycinnamic acid, or a conjugate, analogue or derivative thereof.

In some embodiments, an anti-microbial agent comprises an anti-bacterial agent. In some embodiments, an anti-bacterial agent comprises a polyketide antibiotic. In some embodiments, polyketide antibiotic comprises bacillaene, dihydrobacillaene, or a combination thereof. In some embodiments, said anti-microbial agent comprises an anti-fungal agent.

In another embodiment, the present invention provides a method for enhancing extracellular matrix production in bacteria, the method comprising administering to said bacteria a composition comprising a hydroxycinnamic acid, or a conjugate, analogue or derivative thereof.

In some embodiments, the bacteria are B. subtilis. In some embodiments, said bacteria are in a biofilm. In some embodiments, said bacteria are in a bacterial culture. In some embodiments, said bacteria are present on a root of a plant. In some embodiments, the composition is indirectly administered to said bacteria. In some embodiments, said bacteria are present on an organ of a plant and said composition is administered to said plant.

In another embodiment, the present invention provides a method for inducing, enhancing, or maintaining biofilm formation by a beneficial rhizobacteria on a plant or a part thereof, the method comprising the step of administering to the plant an effective amount of a composition comprising a hydroxycinnamic acid, or a conjugate, analogue or derivative thereof. In some embodiments, said biofilm comprises a B. subtilis biofilm.

In another embodiment, the present invention provides a method for increasing resistance to disease in a plant, the method comprising administering to the plant a composition comprising a hydroxycinnamic acid, or an analogue or derivative thereof.

In another embodiment, the present invention provides a method of treating a disease in a plant, the method comprising administering to a plant in need thereof a composition comprising a hydroxycinnamic acid, or a conjugate, analogue or derivative thereof. In some embodiments, said disease is characterized by an infestation or infection by a plant pathogen.

In another embodiment, the present invention provides a method for suppressing the growth or survival of a biological agent on or near a plant, the method comprising administering to the plant a composition comprising a hydroxycinnamic acid, or an analogue or derivative thereof. In some embodiments, said biological agent comprises a plant pathogen. In some embodiments, said plant pathogen comprises a fungal pathogen.

In some embodiments, said fungal pathogen comprises Rhizoctonia solani. In some embodiments, said plant pathogen comprises a bacterial pathogen. In some embodiments, said bacterial pathogen comprises Pseudomonas syringae. In some embodiments, said bacterial pathogen comprises Xanthomonas campestris.

In some embodiments, said biological agent comprises a non-pathogenic bacterial species. In some embodiments, said non-pathogenic bacterial species comprises S. plymuthica, P. chlororaphis, or both. In some embodiments, said plant comprises a crop plant. In some embodiments, said crop plant comprises a Brassicaceae family plant. In some embodiments, said Brassicaceae family plant comprises Brassica oleracea (cabbage, broccoli, Brussels sprouts, kohlrabi, or cauliflower), Brassica rapa (turnip), Brassica napus (rapeseed), mustard, Raphanus raphanistrum (radish), Armoracia rusticana (horseradish), Lepidium sativum (cress), Eutrema japonicum (wasabi), Eruca sativa (arugula), Nasturtium officinale (watercress), Brassica juncea, (brown mustard), or canola.

In some embodiments, said crop plant comprises a Solanum lycopersicum (tomato), Solanum tuberosum (potato), Zea mays (corn), or Triticum aestivum (wheat). In some embodiments, said composition is administered to said plant by soil injection, soil drenching, injection ofthe composition into plant vasculature, or a combination thereof. In some embodiments, said composition is administered to the root of said plant.

In some embodiments, said hydroxycinnamic acid, or a conjugate, analogue or derivative thereof is selected from a group comprising caffeic acid, 7-galloylcatechin, aesculetin, and chlorogenic acid. In some embodiments, said composition comprising hydroxycinnamic acid further comprises a polyamine. In some embodiments, said hydroxycinnamic acid derivative comprises an amide derivative comprising a polyamine.

In some embodiments, said polyamine comprises putrescine. In some embodiments, said hydroxycinnamic acid amide derivative comprises caffeoyl putrescine.

In another embodiment, the present invention provides a method for isolating caffeoyl putrescine from the root or root exudate of a plant, the method comprising: (a) obtaining root exudate from said plant; (b) fractionating and purifying said exudate; (c) dansylating amine groups in the fraction of said exudate that blocks B. subtilis biofilm dispersal; and (d) resolving dansylated compounds from selected fractions using thin layer chromatography, thereby isolating caffeoyl putrescine from the root or root exudate of said plant.

In some embodiments, fractionation of step (b) comprises HPLC chromatography followed by liquid chromatography-mass spectrometry (LC-MS). In some embodiments, the fraction blocking B. subtilis biofilm dispersal is tested in step (c) with a pellicle development assay. In some embodiments, the dansylation in step (c) comprises derivatization of amine groups with dansyl chloride. In some embodiments said plant was optionally grown under iron-deficient conditions.

In another embodiment, the present invention provides a composition comprising a mixture of a) a hydroxycinnamic acid or an analogue or derivative of hydroxycinnamic acid and b) a polyamine or an analogue or derivative of a polyamine. In some embodiments, said hydroxycinnamic acid or an analogue or derivative of hydroxycinnamic acid comprising caffeic acid, 7-galloylcatechin, aesculetin, and chlorogenic acid. In some embodiments, said composition comprises caffeic acid, 7-galloylcatechin, aesculetin, or chlorogenic acid at a concentration of 1-150 μM. In some embodiments, said composition comprises caffeic acid, 7-galloylcatechin, aesculetin, or chlorogenic acid at a concentration of 1-5 μM. In some embodiments, said polyamine comprises putrescine.

In some embodiments, composition comprises caffeic acid, 7-galloylcatechin, aesculetin, or chlorogenic acid, and putrescine at a concentration of 1-5 μM each. In some embodiments, said composition further comprises a botanically compatible vehicle.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1L: B. subtilis expands asymmetrically towards S. plymuthica and eliminates its pre-established biofilms in a host and temperature dependent manner. (FIG. 1A) B. subtilis expanding asymmetrically towards the S. plymuthica colony on the third day of incubation, at different distances of inoculation (indicated in cm in the top left corner); scale bar—0.5 cm. (FIGS. 1B, 1C and 1D) Quantification of asymmetrically expansion of B. subtilis biofilm towards S. plymuthica biofilm. The radius ratio, as measured by the radius from the center of the biofilm extending to the S. plymuthica colony, divided by the radius extending to the opposite side of the biofilm, at different distances of inoculation (cm) in (FIG. 1B) motility and chemotaxis mutants and in (FIG. 1C) matrix mutants. Color-coded bar indicates inoculation distances in cm; non-interacting B. subtilis biofilm. (FIG. 1D) Asymmetrical expansion of B. subtilis is assisted by mining dead S. plymuthica cells. The radius ratio of the B. subtilis biofilm at different distances of inoculation (cm) after a three-day interaction with live S. plymuthica cells, or 24 hr S. plymuthica supernatant, or dead S. plymuthica cells. Color-coded bar indicates inoculation distances in cm; non-interacting B. subtilis biofilm. (FIG. 1E) Left panel-images of interactions between S. plymuthica and WT B. subtilis and B. subtilis Δpks after four days of incubation. While the WT strain was able to engulf and eradicate S. plymuthica, the Δpks strain only succeeded in surrounding, but not in killing it; scale bar—0.2 cm. Right panel—The number of colony forming units (CFUs) represents replicative cell count of S. plymuthica after interaction with either another S. plymuthica biofilm (S.p+S.p) as control, WT B. subtilis strain (S.p+B.s) or Δpks strain (S.p+Apks). The co-inoculated interactions were incubated at 30° C. and 23° C. for three and five days, respectively. (FIG. 1F) SEM images of S. plymuthica from the interaction zone between WT B. subtilis (left) and Δpks B. subtilis (right). Interaction with WT B. subtilis resulted in compressed and distorted S. plymuthica cells, while interaction with AΔks B. subtilis resulted in intact S. plymuthica cells. Interactions were analyzed after 48 hours of incubation at 30° C. (FIGS. 1G-1I) Testing the effect of the plant host on interaction of B. subtilis and S. plymuthica. (FIGS. 1G-1H) S. plymuthica CFUs (G) and B. subtilis CFUs (FIG. 1H) were tested after interaction in proximity of 0.8 cm to E. sativa seedling (With plant) and without seedling (No plant) for three and five days at 23° C. S. plymuthica CFUs (FIG. 1G) were obtained after interaction with either another S. plymuthica biofilm (S.p+S.p), WT B. subtilis biofilm (S.p+B.s) or with Δpks B. subtilis biofilm (S.p+Δpks). (FIG. 1I) Ratio of CFUs of each species alone grown with and without the plant. *P-value<0.01, **P-value<0.005 based on a two tailed students' t-test of the entire indicated measurements series. (FIG. 1J) S. plymuthica CFUs interacted with either another S. plymuthica biofilm (S.p+S.p) as control and WT B. subtilis (S.p+B.s) on a MSgg-agar plate pretreated with root exudate or plant growth medium (control) for 5 days at 23° C. (FIG. 1K) Growth (OD₆₀₀) Pₚₖₛ-lux strain treated with root exudate or plant growth medium. (FIG. 1L) Growth-normalized RLU of P_{sdpC}-lux strain treated with root exudate or plant growth medium. Results for (FIG. 1J) and (FIG. 1K) represent averages and standard error of a representative experiment performed with four technical repeats, out of three independent experiments.

FIGS. 2A-2C: B. subtilis eliminates S. plymuthica colony cells following their asymmetric expansion. (FIGS. 2A-2B) The number of colony forming units (CFU) represents (FIG. 2A) B. subtilis or (FIG. 2B) S. plymuthica replicative cells at 24 h (No contact), 48 h (Direct contact), and 72 hr (Covered) after inoculation. Error bars represents ±S. D of 5 biological replicates. **P-value<0.005. The matrix mutants differed from each other by P-value<0.01. (FIG. 2C) Percentage of bacterial CFU at each stage of the interactions. The area of both interacting colonies was divided into three sections: B.s—B. subtilis, int—interaction, and S.p—S. plymuthica. Each section was harvested, sonicated and plated to reveal the number of replicative cells of each specie.

FIGS. 3A:-3H Specialized host-derived metabolites increase PKS activity while B. subtilis biofilms increase plant Induced Systemic Resistance (ISR). (FIG. 3A) Quantification of pks expression (amyE::Pₚₖₛ-gfp) in B. subtilis over time in response to different hydrophobicity fractions of plant root exudate. Expression was measured using fluorescent intensity of GFP fused to the PKS promoter. Color-coded curves indicate ascending methanol concentrations used for consecutive washings of root exudate from Silica-based bonded phase with strong hydrophobicity; darker curve indicates the first flow-through of the loaded root exudate from the silica cartridge. Orange curve indicate the pks expression kinetics without treatment. (FIG. 3B) Quantification of pks expression (amyE::Pₚₖₛ-gfp) in B. subtilis over time in response to plant root metabolites. Expression was measured by fluorescent intensity of GFP fused to the PKS promotor. Color-coded curves indicate 5, 50,100 and 150 μM of the four metabolites: Methyl β-D-Glucopyranosid (upper left), Chromone (upper right), Chlorogenic acid (lower left) and Caffeic acid (lower right). Orange curve indicate the pks expression kinetics without treatment. (FIG. 3C) Fluorescence imaging of pks expression (amyE::Pₚₖₛ-gfp) in a single cell resolution 5 h after B. subtilis was grown in MSgg (control) liquid culture supplemented with 150 μM of Chlorogenic acid and Caffeic acid. Images presented as over-layered channels of TL-phase and EGFP. Scale bar—10 μM. (FIG. 3D) Fluorescent cell count of those that its intensity mean value exceeded arbitrarily threshold of 1000, reflecting highly pks expression (amyE::Pₚₖₛ-gfp). (E-G) The effects of B. subtilis and S. plymuthica on the induced systemic resistance (ISR) to leaves' infection against P. syringae in E. sativa leaves. Pathogenicity evaluation was achieved by measuring the area of the black spots (necrosis tissue) appeared over the leaves 5 days after inoculation. (FIG. 3E) A diagram of the course of the experiment. Biofilms are established on E. sativa plants prior to their challenge with P. syringae. (FIG. 3F) Necrosis area (mm2) in E. sativa leaf caused by the pathogen P. syringae. Pathogenicity was measured in E. sativa seedlings whose roots were inoculated with B. subtilis, S. plymuthica and their combination in ratios of 1:1, 100:1 and 1:100. (FIG. 3G) Either B. subtilis or S. plymuthica cells were inoculated directly on top of the root. *P-value<0.005. Results represent averages and standard error of a representative experiment performed with five technical repeats, out of three independent experiments. (FIG. 3H) Illustration of the mechanisms involved in the interaction between B. subtilis and Gram negative rhizobacteria on a plant root.

FIG. 4: The B. subtilis biofilm extends towards the S. plymuthica colony. Developmental stages of the interaction between B. subtilis and Serratia plymuthica: no contact (day 1, 24 h), semi-circled (day 2, 48 h), and engulfed (day 3, 72 h); scale bar—0.2 cm

FIG. 5: Motility and chemotaxis B. subtilis mutants expand asymmetrically towards Serratia plymuthica. Motility mutants, Δhag and ΔsigD, and chemotaxis mutants, ΔcheA and ΔcheY, expanded asymmetrically towards the S. plymuthica colony, in the same manner as the WT B. subtilis. Interactions were achieved by inoculating B. subtilis near S. plymuthica at different distances and incubated at 30° C. for three days. A quantification of multiple interacting colonies further confirmed that the wild type spread a symmetrically from inoculation distances of 0.5 cm to 1.2 cm (See FIG. 1B).

FIG. 6: Exopolysaccharides are required for asymmetric expansion towards Serratia plymuthica. ΔtasA lacking functional amyloids expanded asymmetrically but not a ΔepsH strain, which is unable to produce the exopolysaccharides (For quantification, see FIG. 1C).

FIG. 7: Extracellular matrix is not essential for the elimination of S. plymuthica colony cells. The number of S. plymuthica replicative cells at 72 hr after inoculation either alone (NT) or with the indicated B. subtilis strains. The two bacteria were inoculated at a distance of 0.3 cm, due to the small colonies formed by matrix mutants, and the analysis of the total number of cells in the interaction was performed as explained in materials and methods.

FIGS. 8A-8B: During biofilm formation, bacillaene is produced by the PKS operon and required the presence of pksX Chromatograms of (FIG. 8A) a WT biofilm colony extract and (FIG. 8B) a ΔpksX colony extract with isopropanol. The peaks at retention times of 8.38 min and 8.59 min in the WT extract represent two isomers of bacillaene, fully absent in the ΔpksX extract.

FIG. 9: B. subtilis eliminates S. plymuthica cells in a pks dependent manner. Percentage of B. subtilis WT or Δpks and S. plymuthica replicative cells, at different sections of the interaction (B.s—B. subtilis, int—interaction, and S.p—S. plymuthica), 48-96 hr after inoculation. *P-value <0.01, **P-value<0.005 based on a two tailed students' t-test of the entire indicated measurements series.

FIG. 10: Extracellular proteases have a role in the elimination of Serratia plymuthica. Indicated Extracellular Proteases mutants were inoculated at a distance of 0.8 cm from S. plymuthica. In the absence of AprE, a clear consumption defect was observed.

FIG. 11: Polyketides synthesis is critical for elimination of Pseudomonas chlororaphis during competition. The number of Pseudomonas chlororaphis replicative cells at 72 hours after co-inoculation with indicated B. subtilis strains.

FIG. 12: Caffeic acid has little or no effect on the growth of B. subtilis. Planktonic growth of wild type grown at 30° C. with shaking in liquid MSgg medium without (untreated) or caffeic acid at indicated concentrations was monitored by measuring OD₆₀₀in a microplate reader. Results are averages of four wells within one experiment and their standard deviations. A representative of three independent experiments is shown.

FIG. 13: B. subtilis supernatant eliminates S. plymuthica cells. Growth curves of S. plymuthica in liquid biofilm medium, supplemented with 50% [volume/volume] of B. subtilis WT or Δpks supernatant collected at 10 hours and 24 hours as indicated. Experiments were performed at 23° C. *P-value<0.01, **P-value<0.005 based on a two tailed students' t-test of the entire indicated measurements series.

FIG. 14: Purification and characterization of the naturally produced RID. Plants were grown under iron starvation over agrophonic beads. Exudates were collected, purified, analyzed for beneficial effects on the plant protector B. subtilis, after derivatization with dansyl chloride, and analyzed by thin-liquid chromatography (TLC).

FIG. 15: Caffeic acid can promote biofilm maintenance of the plant protector B. subtilis. Shown are biofilms of B. subtilis at day 1 (upper panel) and day 5 (bottom panel, broken biofilms are also characterized by brown pigmentation prevented by caffeic acid).

FIG. 16: Caffeic acid and putrescine can promote biofilm maintenance of the plant protector B. subtilis in a low micro-molar concentration. Shown are biofilms of treated and untreated B. subtilis biofilms. 5 μM of caffeic acid-putrescine mixture (2.5 μM each) prevented the breaking down of the biofilm.

FIGS. 17A-17C: Caffeic acid and a caffeic acid-putrescine mixture can promote biofilm maintenance of the plant protector B. subtilis. (FIGS. 17A-17C) B. subtilis 3610 constantly expressing the fluorescent protein mKate2 was maintained on LB plates. 1000 cells either applied or non-applied with the materials indicated were added to tomato plants grown on agrophonic beads. % coverage was calculated based on quantifying 20 fields from the fluorescent microscope using Image J. Three replications were maintained for each assay. SD represents the average of three independent experiments.

FIGS. 18A-18B: Purified RID improved the bioprotection versus pseudomonas syringae achieved by Bacillus. Bacillus strain B. subtilis 3610 (B.S) was used. The bacterial strain was maintained on LB plates. 1000 cells either applied or non-applied with 5 μM of RID were added to tomato plants grown on agrophonic beads. Plants were incubated at 28±2° C. for 15 days. P. syringae strain DC3000 pv tomato (P.S) culture was grown in LB at 37° C. to OD₆₀₀=0.2 to 0.3 (3*10⁸ cells per ml) and diluted to 100 cells (FIG. 18A) 10,000 (FIG. 18B) cells retrospectively (w/v) into the plant media. Plants were further incubated at 28±2° C. for 10 days and the growth of the pathogen was measured and compared with the control. Three replications were maintained for each assay. SD represents the average of three independent experiments. RID improved significantly the duration of protection achieved by Bacillus subtilis and the resistance to high inoculum of infection.

FIG. 19: Purified RID improved the bioprotection versus pseudomonas syringae and rhyzoctonia solani achieved by Bacillus. We used Bacillus strain B. subtilis 3610 (B.S). The bacterial strain was maintained on LB plates. 1000 cells either applied or non-applied with 5 μM of purified RID were added to Arabidopsis thaliana plants grown on MS solid media. Plants were incubated at 26±2° C. for 5 days. P. syringae (P.S) or R. solani (R.S) were grown in TSB at 37° C. to OD₆₀₀=0.2 to 0.3 (3*10⁸ cells per ml) and diluted to 100 cells, or 10,000 cells retrospectively (w/v) into the plant media. Plants were further incubated at 28±2° C. for 5 days and the growth of the pathogen was measured and compared with the control. Three replications were maintained for each assay. SD represents the average of three independent experiments. RID improved significantly protection achieved by Bacillus subtilis.

FIG. 20: Purified RID can promote the development of biofilms formed by B. subtilis strain, lacking iron-uptake systems. Biofilms grown in the presence and absence of RID in B. subtilis 3610 and its derivate (Δ yclQ) mutant in iron uptake pathways (Zawadzka et al., 2009). Untreated pellicles are shown in the top panel. Pellicles applied with purified RID are shown below.

FIG. 21: Synthesis schemes for producing derivatives of caffeic acid conjugates and derivatives thereof. Abbreviations: EDC—1-Ethyl-3-(3-dimethylaminopropyl)carbodiimide; NHS—N-hydroxysuccinimide; DCM—dichloromethane.

FIGS. 22A-22D: Regulation and Function of the PKS operon. (FIG. 22A) Regulatory elements of ₚₖₛ Promoter. (FIG. 22B) Luminescence (RLU) of WT and indicated mutant strains carrying the P_{pksC}-lux construct. The strains have different rates planktonic growth and therefore luminescence is indicated per equivalent optical densities. (FIG. 22C) Luminescence (RLU) ofWT and indicated mutant strains carrying the P_{pksC}-lux construct. Open circles represent the untreated luminescence while the closed full circles represent the same strain applied with root exudate. Results for (B) and (C) represent averages and standard error of a representative experiment performed with four technical repeats, out of three independent experiments. (FIG. 22D) Genome comparisons between Bacillus subtilis 168, Bacillus atrophaeus NRS 1221A, Bacillus amyloliquefaciens L-S60 and Bacillus methylotrophicus JJ-D34 reveals a conservation of the entire PKS operon (pksA-R). In contrast, Bacillus anthracis HYU01 and Bacillus cereus FT9 strains lacked the pks operon altogether. Orthologous genes were tagged by common colors across the different strains.

FIGS. 23A-23B: An ester of caffeic acid induces antibiotic production. (FIG. 23A) Scheme of a screen of plant metabolites for pks induction. (FIG. 23B) Growth-normalized RLU of P_{pksC}-lux strain treated with a small molecule library representative as in A, compared with the treatment in root exudate.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides methods and compositions for improving fitness of a plant. Without wishing to be bound by any particular theory, plant roots harbor many bacterial species that are found in constant competition over space and nutrients. Both the Gram-positive and the Gram-negative bacteria can form beneficial biofilms on plant roots. As used herein, the term “biofilm” refers to an adherent film formed by biological material, e.g., at least one microorganism, that comprises thin layers of ensheathed filamentous microbial colonies on a surface. Without wishing to be bound by any particular theory, it is believed that some of the bacterial species forming biofilm on plant roots enter symbiotic relationship with the host plant, wherein the bacteria are activated by the host plant to produce agents that kill or suppress the growth of other microorganisms, including pathogenic organisms, thereby protecting the host plant from said pathogens.

In one embodiment, the bacteria as described herein produce one or more anti-microbial agents. In another embodiment, the bacteria as described herein produce one or more anti-pathogenic agents.

As used herein, the term “anti-microbial agent” refers to a chemical or other agent that suppresses or kills a target microorganism. In one embodiment, an anti-microbial agent as described herein suppresses the growth of a target pathogenic microorganism, for example, a bacterial pathogen or a fungal pathogen (i.e., exhibits antibacterial or antifungal activity). In another embodiment, an anti-microbial agent as described herein suppresses the growth of a target non-pathogenic miocroorganism, which, in one embodiment, comprises a competing bacterial species. In one embodiment, growth of the micro-organism is suppressed by interfering with the normal growth of target microorganism, for example through inhibiting vital enzymes.

In another embodiment, the term “anti-pathogenic agent” as used herein refers to a chemical or other agent that suppresses or kills a target pathogen.

Accordingly, the present invention provides methods and compositions for improving fitness of a plant through stimulation of production of an anti-microbial agent by bacteria forming a biofilm at the plant roots. As used herein the term “fitness of a plant” refers to one or more of improved resistance to disease; improved ability to defend against disease; reduction of disease symptoms; faster growth; improved crop productivity; improved crop quality; inducing systemic pathogen resistance; treating plant disease; and stimulating the defense mechanism in a plant.

Multiple bacterial species capable of protective biofilm on the plant roots are contemplated as part of the present invention. In one embodiment of the present invention, the suitable bacterial species include bacteria from the genus Bacillus. In one embodiment, suitable bacterial species comprise Bacillus species within the Bacillus subtilis clade. In a particularly preferred embodiment, suitable bacterial species comprise B. pumilus, B. atrophaeus, B. amyloliquefaciens, B. subtilis or B. licheniformis. In one embodiment, the Bacillus species comprises Bacillus subtilis.

Without wishing to be bound by any particular theory, it is believed that host plant promotes growth of beneficial B. subtilis biofilms at the roots through exuding stimulatory compounds. These compounds may function through various mechanisms, including, without limitation, directly stimulating bacterial growth and biofilm formation, increasing coverage area of biofilms on plant's roots, suppressing dispersal of biofilms, and activating B. subtilis-mediated suppression of the growth of competing microorganisms, such as bacteria and fungi, including pathogenic microorganisms.

In one embodiment, the plant-produced stimulatory compound comprises a hydroxycinnamic acid. Numerous hydroxycinnamic acid compounds are known in the art, including, without limitation, α-cyano-4-hydroxycinnamic acid, cichoric acid, cinnamic acid, chlorogenic acid, neochlorogenic acid, diferulic acid, coumaric acid, coumarin, ferulic acid, sinapinic acid, zapotin, 4-O-Caffeoylquinic acid, caffeic acid, 7-galloylcatechin, aesculetin, and chlorogenic acid. Each represents a separate embodiment of the invention.

In one embodiment the plant-produced stimulatory compound comprises caffeic acid having the structure of Formula I:

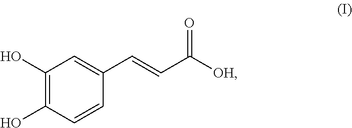 [SMILES: O=C(O)/C=C/c1ccc(O)c(O)c1]

or an analogue, conjugate, or derivative thereof.

In one embodiment the plant-produced stimulatory compound comprises 7-galloylcatechin having the structure of Formula II:

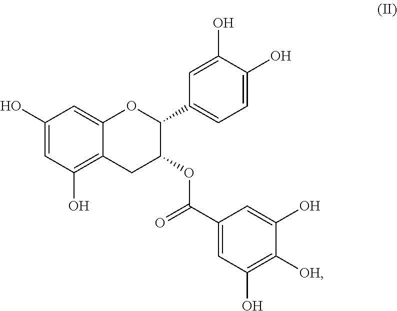 [SMILES: O=C(O[C@@H]1Cc2c(O)cc(O)cc2O[C@@H]1c1ccc(O)c(O)c1)c1cc(O)c(O)c(O)c1]

In one embodiment the plant-produced stimulatory compound comprises aesculetin having the structure of Formula III:

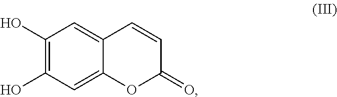 [SMILES: O=c1ccc2cc(O)c(O)cc2o1]

In one embodiment the plant-produced stimulatory compound comprises aesculetin having the structure of Formula IV:

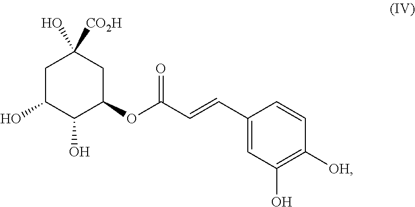 [SMILES: O=C(/C=C/c1ccc(O)c(O)c1)O[C@@H]1C[C@](O)(C(=O)O)C[C@@H](O)[C@H]1O]

In one embodiment, the plant-produced stimulatory compound does not comprise a caffeic acid ester such as rosmarinic acid (RA).

In another embodiment, the plant-produced stimulatory compound comprises a polyamine compound. Numerous polyamine compounds are known in the art, including, without limitation, putrescine, cadaverine, spermidine and spermine. Each represents a separate embodiment of the invention.

In one embodiment the plant-produced stimulatory compound comprises putrescine comprising the structure of Formula V:

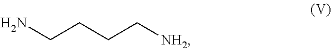

or an analogue, conjugate, or derivative thereof.

In one embodiment, the plant-produced compound comprises a conjugate of a hydroxycinnamic acid compound and a polyamine compound. In one embodiment, the plant-produced compound comprises a conjugate of caffeic acid and putrescine (caffeoyl putrescine) comprising the structure of Formula VI:

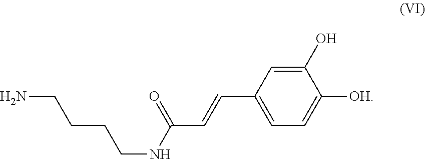 [SMILES: NCCCCNC(=O)/C=C/c1ccc(O)c(O)c1]

In another embodiment, compositions of the present invention and compositions for use in the present invention comprise caffeoyl putrescine. In another embodiment, compositions of the present invention and compositions for use in the present invention comprise an analogue of caffeoyl putrescine. In another embodiment, compositions as described herein comprise a derivative of caffeoyl putrescine. In another embodiment, compositions as described herein comprise a conjugate of an analogue or derivative of caffeic acid and an analogue or derivative of putrescine.

### Compositions

In one embodiment, the compositions of the present invention and the compositions for use in the methods of the present invention comprise an effective amount of a plant-produced stimulatory compound or any combination of plant-produced stimulatory compounds described herein, and a botanically acceptable carrier or vehicle.

In one embodiment, the plant-produced stimulatory compound comprises caffeic acid. In one embodiment, the plant-produced stimulatory compound comprises 7-galloylcatechin. In one embodiment, the plant-produced stimulatory compound comprises aesculetin. In one embodiment, the plant-produced stimulatory compound comprises chlorogenic acid.

In one embodiment, the compositions of the present invention and the compositions for use in the methods of the present invention comprise an effective amount of a hydroxycinnamic acid or derivative or analogue thereof. In another embodiment, the compositions of the present invention comprise a polyamine. In another embodiment, the compositions of the present invention comprise a diamine. In another embodiment, the compositions of the present invention comprise a conjugate of a hydroxycinnamic acid and a polyamine. In another embodiment, the compositions of the present invention comprise a conjugate of a hydroxycinnamic acid and a diamine. In one embodiment, the hydroxycinnamic acid is caffeic acid. In one embodiment, the hydroxycinnamic acid is 7-galloylcatechin. In one embodiment, the hydroxycinnamic acid is aesculetin. In one embodiment, the hydroxycinnamic acid is chlorogenic acid.

In one embodiment, the composition of this invention comprises a hydroxycinnamic acid conjugate compound represented by the structure of Formula VII:

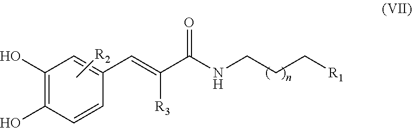

wherein:
R₁ is alkyl, aryl, alkenyl, alkynyl, amine, halide, nitro, cyano, —PO₄H₂, —PO₄HNa, PO₄Na₂, CO₂K or CO₂Na;
   R₂ is H, alkyl, halide, nitro, hydroxy, cyano, —PO₄H₂, —PO₄HNa, PO₄Na₂, CO₂K or CO₂Na
   R₃ is H, alkyl, alkenyl, alkynyl, nitro, cyano or halide;
   and
   n is 1-10.

In one embodiment, the composition of this invention comprises a hydroxycinnamic acid conjugate compound represented by the structure of Formula VIII:

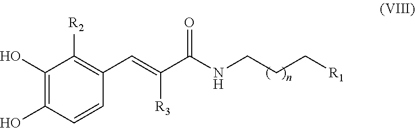

wherein:
R₁ is alkyl, aryl, alkenyl, alkynyl, amine, halide, nitro, cyano, —PO₄H₂, —PO₄HNa, PO₄Na₂, CO₂K or CO₂Na;
R₂ is H, alkyl, halide, nitro, hydroxy, cyano, —PO₄H₂, —PO₄HNa, PO₄Na₂, CO₂K or CO₂Na;
R₃ is H, alkyl, alkenyl, alkynyl, nitro, cyano or halide;
and
n is 1-10.

In one embodiment, the composition of this invention comprises a hydroxycinnamic acid conjugate compound represented by the structure of Formula IX:

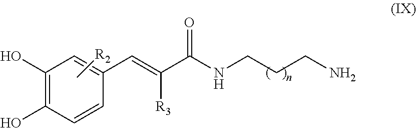

wherein:
R₂ is H, alkyl, halide, nitro, hydroxy, cyano, —PO₄H₂, —PO₄HNa, PO₄Na₂, CO₂K or CO₂Na;
R₃ is H, alkyl, alkenyl, alkynyl, nitro, cyano or halide;
and
n is 1-10.

In one embodiment, the composition of this invention comprises a hydroxycinnamic acid derivative compound represented by the structure of Formula X:

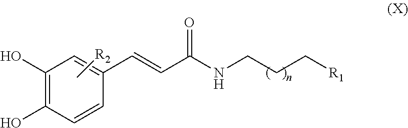

wherein:
R₁ is alkyl, aryl, alkenyl, alkynyl, amine, halide, nitro, cyano, —PO₄H₂, —PO₄HNa, PO₄Na₂, CO₂K or CO₂Na;
R₂ is H, alkyl, halide, nitro, hydroxy, cyano, —PO₄H₂, —PO₄HNa, PO₄Na₂, CO₂K or CO₂Na;
and
n is 1-10.

In one embodiment, the composition of this invention comprises a hydroxycinnamic acid derivative compound represented by the structure of Formula XI:

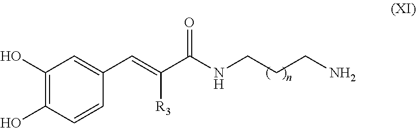

wherein:
R₃ is H, alkyl, alkenyl, alkynyl, nitro, cyano or halide;
and
n is 1-10.

In one embodiment, the composition of this invention comprises a hydroxycinnamic acid derivative compound represented by the structure of Formula XII:

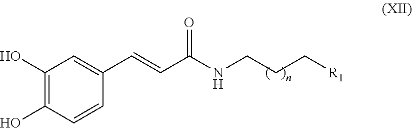

wherein:
R₁ is alkyl, aryl, alkenyl, alkynyl, amine, halide, nitro, cyano, —PO₄H₂, —PO₄HNa, PO₄Na₂, CO₂K or CO₂Na; and
n is 1-10.

In one embodiment, the composition of this invention comprises a hydroxycinnamic acid derivative compound represented by the structure of Formula XIII:

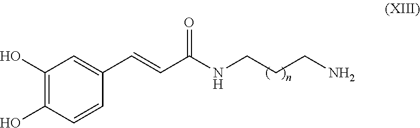

wherein:
n is 1-10.

In another embodiment, the compositions of the present invention comprise a botanically acceptable carrier or vehicle. In one embodiment, the hydroxycinnamic acid, polyamine, or hydroxycinnamic acid and polyamine conjugate are effective in the methods described herein, such as enhancing the production of an anti-microbial agent in bacteria, enhancing extracellular matrix production in bacteria, activating the pks gene operon in bacteria having apks gene operon, increasing resistance to disease in a plant, treating a disease in a plant, suppressing the growth or survival of a biological agent on or near a plant, stimulating the defense mechanism of a plant, accelerating growth of a plant, improving crop productivity, improving crop quality, etc.

In another embodiment, the compositions of the present invention and the compositions for use in the methods of the present invention comprise a composition for administration to plants comprising caffeic acid, putrescine, a caffeic acid-putrescine conjugate, a mixture of caffeic acid and a polyamine, an analogue or derivative of caffeic acid, analogue or derivative of putrescine, analogue or derivative of a caffeic acid-putrescine conjugate, 7-galloylcatechin, aesculetin, chlorogenic acid or any combination thereof, and a botanically compatible vehicle. In another embodiment, the compositions of the present invention and the compositions for use in the methods of the present invention comprise a plant-based composition comprising caffeic acid, putrescine, a caffeic acid-putrescine conjugate, 7-galloylcatechin, aesculetin, chlorogenic acid, analogue thereof, derivative thereof, or any combination thereof, and a botanically compatible vehicle.

In another embodiment, compositions of the present invention and compositions for use in the present invention comprise a hydroxycinnamic acid compound, as described hereinabove. In one embodiment, the hydroxycinnamic acid is caffeic acid. In one embodiment, the hydroxycinnamic acid is 7-galloylcatechin. In one embodiment, the hydroxycinnamic acid is aesculetin. In one embodiment, the hydroxycinnamic acid is chlorogenic acid.

In one embodiment, hydroxycinnamic acid compounds for use in the compositions and methods of the present invention comprise α-cyano-4-hydroxycinnamic acid, cichoric acid, cinnamic acid, chlorogenic acid, neochlorogenic acid, diferulic acid, coumaric acid, coumarin, ferulic acid, sinapinic acid, zapotin, 4-O-Caffeoylquinic acid, caffeic acid, 7-galloylcatechin, aesculetin, or a combination thereof.

In another embodiment, compositions of the present invention and compositions for use in the present invention comprise a polyamine, as described hereinabove.

In one embodiment, amines and polyamines for use in the compositions and methods of the present invention comprise 1,2-Diamino-3,5-dimethylbenzene; tert-Butyl 2-aminoethyl(ethyl)carbamate hydrochloride; Polyethylenimine; Poly(pyromellitic dianhydride-co-4,4′-oxydianiline), amic acid solution; Poly(3,3′,4,4′-benzophenonetetracarboxylic dianhydride-co-4,4′-oxydianiline/1,3-phenylenediamine), amic acid solution; Spermine dehydrate; N-Boc-cadaverine; Spermine; Aminoguanidine bicarbonate; N-(3-Aminopropyl)-2-pyrrolidinone; 1,1,4,7,10,10-Hexamethyltriethylenetetramine; spermine tetrahydrochloride; N1-Boc-2,2′-iminodiethylamine; Spermidine; L-Ornithine monohydrochloride; or any combination thereof.

In another embodiment, the present invention provides a composition comprising a) caffeic acid or an analogue or derivative of caffeic acid and b) putrescine or an analogue or derivative of putrescine.

In another embodiment, the compositions of the present invention comprise a mixture of a hydroxycinnamic acid and a polyamine. In another embodiment, the present invention provides a composition comprising a mixture of a) a hydroxycinnamic acid or an analogue or derivative of hydroxycinnamic acid and b) a polyamine or an analogue or derivative of a polyamine.

In another embodiment, compositions of the present invention and compositions for use in the present invention comprise amide derivatives of hydroxycinnamic acid compounds, analogues of hydroxycinnamic acid compounds, derivatives of hydroxycinnamic acid compounds, conjugates of hydroxycinnamic acid compounds, or any combination thereof.

In another embodiment, compositions of the present invention and compositions for use in the present invention comprise amide derivatives of caffeic acid, analogues of caffeic acid, derivatives of caffeic acid, conjugates of caffeic acid, or any combination thereof.

In another embodiment, compositions of the present invention and compositions for use in the present invention comprise amide derivatives of 7-galloylcatechin, analogues of 7-galloylcatechin, derivatives of 7-galloylcatechin, conjugates of 7-galloylcatechin, or any combination thereof.

In another embodiment, compositions of the present invention and compositions for use in the present invention comprise amide derivatives of aesculetin, analogues of aesculetin, derivatives of aesculetin, conjugates of aesculetin, or any combination thereof.

In another embodiment, compositions of the present invention and compositions for use in the present invention comprise amide derivatives of chlorogenic acid, analogues of chlorogenic acid, derivatives of chlorogenic acid, conjugates of chlorogenic acid, or any combination thereof.

In another embodiment, compositions of the present invention and compositions for use in the present invention comprise analogues of a polyamine, derivatives of a polyamine, or any combination thereof.

In another embodiment, compositions of the present invention and compositions for use in the present invention comprise analogues of putrescine, derivatives of putrescine, or any combination thereof. Each represents a separate embodiment of the invention.

In another embodiment, the compositions of the present invention and the compositions for use in the methods of the present invention comprise a conjugate of caffeic acid and putrescine or an analogue thereof, derivative thereof. In another embodiment, the compositions of the present invention and the compositions for use in the methods of the present invention comprise one or more polyamines, one or more amines, or both. In another embodiment, the compositions of the present invention and the compositions for use in the methods of the present invention comprise an analogue of caffeic acid or derivative thereof, an analogue of putrescine or derivative thereof, or any combination thereof. In one embodiment, the compositions further comprise a botanically acceptable carrier or vehicle.

In some embodiments, the term “analog” refers to structurally or, in another embodiment, functionally related compound.

In some embodiments, the term “derivative” refers to a compound that is derived from a similar compound by a chemical process.

In some embodiments, an “alkyl” group refers to a saturated aliphatic hydrocarbon, including straight-chain or branched-chain. In some embodiments, alkyl is linear or branched. In another embodiment, alkyl is optionally substituted linear or branched. In another embodiment, alkyl is methyl. In another embodiment alkyl is ethyl. In some embodiments, the alkyl group has 1-20 carbons. In another embodiment, the alkyl group has 1-8 carbons. In another embodiment, the alkyl group has 1-7 carbons. In another embodiment, the alkyl group has 1-6 carbons. In another embodiment, non-limiting examples of alkyl groups include methyl, ethyl, propyl, isobutyl, butyl, pentyl or hexyl. In another embodiment, the alkyl group has 1-4 carbons. In another embodiment, the alkyl group may be optionally substituted by one or more groups selected from halide, hydroxy, alkoxy, carboxylic acid, aldehyde, carbonyl, amido, cyano, nitro, amino, alkenyl, alkynyl, arylazide, epoxide, ester, acyl chloride and thiol.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or — or —C(O)NH₂.

In some embodiments, the term “alkenyl” used herein refers to any alkyl group wherein at least one carbon-carbon double bond (C═C) is found. In another embodiment, the carbon-carbon double bond is found in one terminal ofthe alkenyl group. In another embodiment, the carbon-carbon double bond is found in the middle of the alkenyl group. In another embodiment, more than one carbon-carbon double bond is found in the alkenyl group. In another embodiment, three carbon-carbon double bonds are found in the alkenyl group. In another embodiment, four carbon-carbon double bonds are found in the alkenyl group. In another embodiment, five carbon-carbon double bonds are found in the alkenyl group. In another embodiment, the alkenyl group comprises a conjugated system of adjacent alternating single and double carbon-carbon bonds. In another embodiment, the alkenyl group is a cycloalkenyl, wherein “cycloalkenyl” refers to a cycloalkyl comprising at least one double bond.

In some embodiments, the term “alkynyl” used herein refers to any alkyl group wherein at least one carbon-carbon triple bond (C≡C) is found. In another embodiment, the carbon-carbon triple bond is found in one terminal of the alkynyl group. In another embodiment, the carbon-carbon triple bond is found in the middle of the alkynyl group. In another embodiment, more than one carbon-carbon triple bond is found in the alkynyl group. In another embodiment, three carbon-carbon triple bonds are found in the alkynyl group. In another embodiment, four carbon-carbon triple bonds are found in the alkynyl group. In another embodiment, five carbon-carbon triple bonds are found in the alkynyl group. In another embodiment, the alkynyl group comprises a conjugated system. In another embodiment, the conjugated system is of adjacent alternating single and triple carbon-carbon bonds. In another embodiment, the conjugated system is of adjacent alternating double and triple carbon-carbon bonds. In another embodiment, the alkynyl group is a cycloalkynyl, wherein “cycloalkynyl” refers to a cycloalkyl comprising at least one triple bond.

In some embodiments, the term “amine” used herein refers to any N(alkyl)₂, N(aryl)₂, N(alkyl)(aryl), NH(alkyl), NH(aryl), wherein alkyl and aryl are as defined herein above. Each possibility represents a separate embodiment of this invention.

In some embodiments, the term “halide” used herein refers to any substituent of the halogen group (group 17). In another embodiment, halide is flouride, chloride, bromide or iodide. In another embodiment, halide is fluoride. In another embodiment, halide is chloride. In another embodiment, halide is bromide. In another embodiment, halide is iodide.

In some embodiments, R¹ comprises alkyl, aryl, alkenyl, alkynyl, amine, halide, nitro, cyano, —PO₄H₂, —PO₄HNa, PO₄Na₂, CO₂K or CO₂Na, wherein alkyl, aryl, alkenyl, alkynyl, amine and halide are as defined hereinabove. Each possibility represents a separate embodiment of this invention.

In some embodiments, R² comprises H, halide, nitro, cyano, —PO₄H₂, —PO₄HNa, PO₄Na₂, CO₂K or CO₂Na, wherein halide is as defined hereinabove. Each possibility represents a separate embodiment of this invention.

In some embodiments, R³ comprises H, alkyl, alkenyl, alkynyl, nitro, cyano or halide, wherein alkyl, alkenyl, alkynyl and halide are as defined hereinabove. Each possibility represents a separate embodiment of this invention.

In one embodiment, the term “conjugate” refers to a substance formed from the joining together of two parts. Representative conjugates in accordance with the present invention include those formed by the joining together of at least two molecules, for example via a covalent bond, e.g. caffeoyl putrescine, formed from the conjugation of caffeic acid and putrescine.

In another embodiment, the term “botanically acceptable carrier/vehicle” or “botanically compatible carrier/vehicle,” as used herein, refers to any vehicle, in liquid, solid or gaseous form which is compatible with use on a living plant and is convenient to contain a substance or substances for application of the substance or substances to the plant, its leaves or root system, its seeds, the soil surrounding the plant, or for injection into the trunk, or any known method of application of a compound to a living plant, preferably a crop plant, for example a citrus tree, or corn, soybean or tomato plant. In one embodiment, administration is on or near one or more plant roots. Useful vehicles can include any known in the art, for example liquid vehicles, including aqueous vehicles, such as water, solid vehicles such as powders, granules or dusts, or gaseous vehicles such as air or vapor. Any vehicle which can be used with known devices for soaking, drenching, injecting into the soil or the plant, spraying, dusting, or any known method for applying a compound to a plant, is contemplated for use with embodiments of the invention. Typical carriers and vehicles contain inert ingredients such as fillers, bulking agents, buffers, preservatives, anti-caking agents, pH modifiers, surfactants, soil wetting agents, adjuvants, and the like. Suitable carriers and vehicles within this definition also can contain additional active ingredients such as plant defense inducer compounds, nutritional elements, fertilizers, pesticides, and the like. In a particular embodiment, the botanically acceptable vehicle pertains to a vehicle component, or vehicle formulation, that is not found in nature. In another embodiment, the botanically acceptable vehicle may pertain to a vehicle found in nature, but where the vehicle and the bacteria strain(s) are not mixed or combined together in nature. In one embodiment, the vehicle is a non-naturally occurring vehicle.

In one embodiment, the present invention provides administration of an effective amount of a composition as described herein or, alternatively, administration of a composition comprising an effective amount of a composition comprising caffeic acid, putrescine, a caffeic acid-putrescine conjugate, 7-galloylcatechin, aesculetin, chlorogenic acid, or any combination thereof. As used herein, the term “effective amount” refers, in one embodiment, to an amount that is capable of preventing, ameliorating, and/or treating a pathological condition described herein. In another embodiment, the term “effective amount” refers to an initial amount of an active ingredient in the composition that is sufficient to achieve the optimal fmal concentration of said active ingredient upon administering of the compositions to a plant using methods described herein. This effective initial amount depends on multiple factors, including the target plant, the nature of the disease the method of administration, etc., and methods of determining the effective initial amount are well within competence of one of the ordinary skill in the art.

The formulations according to various embodiments may further include at least one sticking agent. A sticking agent comprises a compound that has as at least one of its characteristics the ability to adhere to a surface structure of a plant or to at least one other component in a given formulation. Suitable sticking agents include, but are not limited to yucca plant extracts, Kaolin clay; fine wet-able powders, and the like.

The formulations according to various embodiments may also include at least one agent or compound that at least helps to protect components of a given formulation from the damaging effects ofultraviolet (UV) radiation, or from rapid desiccation. These compounds include, but are not limited to fine clays, Kaolin clay, aluminum oxide, zinc oxide, aluminum silicate, and the like.

The formulations according to various embodiments may furthermore include at least one wetting agent. A wetting agent promotes the dispersal of the formulation in an aqueous environment. Wetting agents may also promote a more even, more efficient, spreading of various components in the formulation onto above-ground plant structures including, but not limited to, leaves, stems, petioles, bark, blossoms, fruits, and the like. Suitable wetting agents include, but are not limited to yucca plant extract.

Compositions according to embodiments of the invention may be formulated as an emulsifiable concentrate(s), suspension concentrate(s), directly sprayable or dilutable solution(s), coatable paste(s), dilute emulsion(s), wettable powder(s), soluble powder(s), dispersible powder(s), dust(s), granule(s) or capsule(s).

### Applications

In one embodiment, the present invention provides novel uses for the plant-produced compounds, or stimulatory metabolites thereof, as described herein. In another embodiment, the present invention provides novel uses for analogues and derivatives of the plant-produced compounds, or stimulatory metabolites thereof, as described herein.

Accordingly the present invention provides, in one embodiment, a method for inducing systemic pathogen resistance in a plant comprising administering to the plant an effective amount of a composition comprising a plant-produced stimulatory compound. In another embodiment, the present invention provides a method for inducing systemic pathogen resistance in a plant comprising administering to the plant an effective amount of a hydroxycinnamic acid, a polyamine, a hydroxycinnamic acid-polyamine conjugate, or any combination thereof. In another embodiment, the present invention provides a method for inducing systemic pathogen resistance in a plant comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inducing systemic pathogen resistance in a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inducing systemic pathogen resistance in a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inducing, improving or increasing resistance to disease in a plant through administering to the plant an effective amount of a composition comprising a plant-produced stimulatory compound. In another embodiment, the present invention provides a method for inducing, improving or increasing resistance to disease in a plant comprising administering to the plant an effective amount of a hydroxycinnamic acid, a polyamine, a hydroxycinnamic acid-polyamine conjugate, or any combination thereof. In another embodiment, the present invention provides a method for inducing, improving or increasing resistance to disease in a plant comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inducing, improving or increasing resistance to disease in a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inducing, improving or increasing resistance to disease in a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for treating plant disease comprising administering to the plant an effective amount of a composition comprising a plant-produced stimulatory compound. In a further embodiment, the present invention provides a method for treating plant disease comprising administering to the plant an effective amount of a composition comprising a hydroxycinnamic acid, a polyamine, a hydroxycinnamic acid-polyamine conjugate, or any combination thereof. In a further embodiment, the present invention provides a method for treating plant disease comprising administering to the plant an effective amount of a composition comprising an analogue or derivative of hydroxycinnamic acid, polyamine, hydroxycinnamic acid-polyamine conjugate, or any combination thereof. In a further embodiment, the present invention provides a method for treating plant disease comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In a further embodiment, the present invention provides a method for treating plant disease comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for stimulating the defense mechanism of a plant comprising administering to the plant an effective amount of a composition comprising a plant-produced stimulatory compound. In yet a further embodiment, the present invention provides a method for stimulating the defense mechanism of a plant comprising administering to the plant an effective amount of a composition comprising a hydroxycinnamic acid, a polyamine, a hydroxycinnamic acid-polyamine conjugate, or any combination thereof. In yet a further embodiment, the present invention provides a method for stimulating the defense mechanism of a plant comprising administering to the plant an effective amount of a composition comprising an analogue or derivative of hydroxycinnamic acid, polyamine, hydroxycinnamic acid-polyamine conjugate, or any combination thereof. In yet a further embodiment, the present invention provides a method for stimulating the defense mechanism of a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet a further embodiment, the present invention provides a method for stimulating the defense mechanism of a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inducing, enhancing, or maintaining biofilm formation by a beneficial rhizobacteria on a plant or a part thereof, the method comprising the step of administering to the plant an effective amount of a composition comprising a plant-produced stimulatory compound. In yet a further embodiment, the present invention provides a method for inducing, enhancing, or maintaining biofilm formation by a beneficial rhizobacteria on a plant or a part thereof, the method comprising the step of administering to the plant an effective amount of a composition comprising a hydroxycinnamic acid, a polyamine, a hydroxycinnamic acid-polyamine conjugate, or any combination thereof. In yet a further embodiment, the present invention provides a method for inducing, enhancing, or maintaining biofilm formation by a beneficial rhizobacteria on a plant or a part thereof, the method comprising the step of administering to the plant an effective amount of a composition comprising an analogue or derivative of hydroxycinnamic acid, polyamine, hydroxycinnamic acid-polyamine conjugate, or any combination thereof. In yet a further embodiment, the present invention provides a method for inducing, enhancing, or maintaining biofilm formation by a beneficial rhizobacteria on a plant or a part thereof, the method comprising the step of administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet a further embodiment, the present invention provides a method for inducing, enhancing, or maintaining biofilm formation by a beneficial rhizobacteria on a plant or a part thereof, the method comprising the step of administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for accelerating growth of a plant comprising administering to the plant an effective amount of a composition comprising a plant-produced stimulatory compound. In yet a further embodiment, the present invention provides a method for accelerating growth of a plant comprising administering to the plant an effective amount of a composition comprising a hydroxycinnamic acid, a polyamine, a hydroxycinnamic acid-polyamine conjugate, or any combination thereof. In yet a further embodiment, the present invention provides a method for accelerating growth of a plant comprising administering to the plant an effective amount of a composition comprising an analogue or derivative of hydroxycinnamic acid, polyamine, hydroxycinnamic acid-polyamine conjugate, or any combination thereof. In yet a further embodiment, the present invention provides a method for accelerating growth of a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet a further embodiment, the present invention provides a method for accelerating growth of a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for reducing disease symptoms in a plant comprising administering to the plant an effective amount of a composition comprising a plant-produced stimulatory compound. In yet another embodiment, the present invention provides a method for reducing disease symptoms in a plant comprising administering to the plant an effective amount of a composition comprising a hydroxycinnamic acid, a polyamine, a hydroxycinnamic acid-polyamine conjugate, or any combination thereof. In yet another embodiment, the present invention provides a method for reducing disease symptoms in a plant comprising administering to the plant an effective amount of a composition comprising an analogue or derivative of hydroxycinnamic acid, polyamine, hydroxycinnamic acid-polyamine conjugate, or any combination thereof. In yet another embodiment, the present invention provides a method for reducing disease symptoms in a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet another embodiment, the present invention provides a method for reducing disease symptoms in a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In one embodiment, a plant pathogen is the cause or source of the plant disease. In one embodiment, the plant disease is an infestation or infection.

In one embodiment, a plant disease as described herein is characterized by an infestation or infection by a plant pathogen.

In another embodiment, the present invention provides a method for suppressing the growth or survival of a biological agent on or near a plant, the method comprising administering to the plant a composition comprising a plant-produced stimulatory compound. In another embodiment, the present invention provides a method for suppressing the growth or survival of a biological agent on or near a plant, the method comprising administering to the plant a composition comprising a hydroxycinnamic acid, a polyamine, a hydroxycinnamic acid-polyamine conjugate, or any combination thereof. In another embodiment, the present invention provides a method for suppressing the growth or survival of a biological agent on or near a plant, the method comprising administering to the plant a composition comprising an analogue or derivative of hydroxycinnamic acid, polyamine, hydroxycinnamic acid-polyamine conjugate, or any combination thereof. In another embodiment, the present invention provides a method for suppressing the growth or survival of a biological agent on or near a plant, the method comprising administering to the plant a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for suppressing the growth or survival of a biological agent on or near a plant, the method comprising administering to the plant an analogue or derivative of caffeic acid, putrescine, caffeoyl putrescine, 7-galloylcatechin, aesculetin, chlorogenic acid, or any combination thereof.

In one embodiment, a biological agent as described herein comprises a bacterium, virus, protozoan, parasite, or fungus. In one embodiment, the biological agent is pathogenic to a plant of interest. In another embodiment, the biological agent is non-pathogenic to a plant of interest. In one embodiment, a biological agent as described herein comprises a plant pathogen. In one embodiment, the plant pathogen comprises a fungal pathogen. In one embodiment, the fungal pathogen is Rhizoctonia solani. In another embodiment, the fungal pathogen is Fusarium graminearum. In another embodiment, the plant pathogen comprises a bacterial pathogen. In one embodiment, the bacterial pathogen is Pseudomonas syringae. In another embodiment, the bacterial pathogen is Xanthomonas campestris. In another embodiment, a biological agent as described herein comprises a non-pathogenic species, which, in one embodiment, comprises a bacterial species. In one embodiment, the bacterial species forms a beneficial biofilm. In one embodiment, the bacterial species comprises P. chlororaphis. In one embodiment, the bacterial species comprises S. plymuthica.

In another embodiment, the present invention provides a method for improving fitness of a plant comprising administering to the plant an effective amount of a composition comprising a plant-produced stimulatory compound. In an additional embodiment, the present invention provides a method for improving fitness of a plant comprising administering to the plant an effective amount of a composition comprising a hydroxycinnamic acid, a polyamine, a hydroxycinnamic acid-polyamine conjugate, or any combination thereof. In an additional embodiment, the present invention provides a method for improving fitness of a plant comprising administering to the plant an effective amount of a composition comprising an analogue or derivative of hydroxycinnamic acid, polyamine, hydroxycinnamic acid-polyamine conjugate, or any combination thereof. In an additional embodiment, the present invention provides a method for improving fitness of a plant comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In an additional embodiment, the present invention provides a method for improving fitness of a plant comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof. In one embodiment, improving fitness of a plant comprises one or more of faster growth, improved crop productivity, or improved crop quality.

In another embodiment, the present invention provides a method for improving crop productivity comprising administering to one or more plants of said crop an effective amount of a composition comprising a plant-produced stimulatory compound. In yet a further embodiment, the present invention provides a method for improving crop productivity comprising administering to one or more plants of said crop an effective amount of a composition comprising a hydroxycinnamic acid, a polyamine, a hydroxycinnamic acid-polyamine conjugate, or any combination thereof. In yet a further embodiment, the present invention provides a method for improving crop productivity comprising administering to one or more plants of said crop an effective amount of a composition comprising an analogue or derivative of hydroxycinnamic acid, polyamine, hydroxycinnamic acid-polyamine conjugate, or any combination thereof. In yet a further embodiment, the present invention provides a method for improving crop productivity comprising administering to one or more plants of said crop an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet a further embodiment, the present invention provides a method for improving crop productivity comprising administering to one or more plants of said crop an analogue or derivative of caffeic acid, putrescine, caffeoyl putrescine, 7-galloylcatechin, aesculetin, chlorogenic acid, or any combination thereof.

In another embodiment, the present invention provides a method for improving crop quality comprising administering to one or more plants of said crop an effective amount of a composition comprising a plant-produced stimulatory compound. In yet a further embodiment, the present invention provides a method for improving crop quality comprising administering to one or more plants of said crop an effective amount of a composition comprising a hydroxycinnamic acid, a polyamine, a hydroxycinnamic acid-polyamine conjugate, or any combination thereof. In yet a further embodiment, the present invention provides a method for improving crop quality comprising administering to one or more plants of said crop an effective amount of a composition comprising an analogue or derivative of hydroxycinnamic acid, polyamine, hydroxycinnamic acid-polyamine conjugate, or any combination thereof. In yet a further embodiment, the present invention provides a method for improving crop quality comprising administering to one or more plants of said crop an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In yet a further embodiment, the present invention provides a method for improving crop quality comprising administering to one or more plants of said crop an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inducing formation of a root-associated bacterial biofilm comprising administering to the plant an effective amount of a composition comprising a plant-produced stimulatory compound. In another embodiment, the present invention provides a method for inducing formation of a root-associated bacterial biofilm comprising administering to the plant an effective amount of a composition comprising hydroxycinnamic acid, a polyamine, a hydroxycinnamic acid-polyamine conjugate, or any combination thereof. In another embodiment, the present invention provides a method for inducing formation of a root-associated bacterial biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inducing formation of a root-associated bacterial biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inducing formation of a root-associated bacterial biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inducing formation of a root-associated B subtilis biofilm comprising administering to the plant an effective amount of a composition comprising a plant-produced stimulatory compound. In another embodiment, the present invention provides a method for inducing formation of a root-associated B subtilis biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for inducing formation of a root-associated B subtilis biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inducing formation of a root-associated B subtilis biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inducing formation of a root-associated B subtilis biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for increasing coverage of a root-associated bacterial biofilm comprising administering to the plant an effective amount of a composition comprising a plant-produced stimulatory compound. In another embodiment, the present invention provides a method for increasing coverage of a root-associated bacterial biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for increasing coverage of a root-associated bacterial biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for increasing coverage of a root-associated bacterial biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for increasing coverage of a root-associated bacterial biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for increasing coverage of a root-associated B subtilis biofilm comprising administering to the plant an effective amount of a composition comprising a plant-produced stimulatory compound. In another embodiment, the present invention provides a method for increasing coverage of a root-associated B subtilis biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for increasing coverage of a root-associated B subtilis biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for increasing coverage of a root-associated B subtilis biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for increasing coverage of a root-associated B subtilis biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for stabilizing a root-associated bacterial biofilm comprising administering to the plant an effective amount of a composition comprising a plant-produced stimulatory compound. In another embodiment, the present invention provides a method for stabilizing a root-associated bacterial biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for stabilizing a root-associated bacterial biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for stabilizing a root-associated bacterial biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for stabilizing a root-associated bacterial biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for stabilizing a root-associated B subtilis biofilm comprising administering to the plant an effective amount of a composition comprising a plant-produced stimulatory compound. In another embodiment, the present invention provides a method for stabilizing a root-associated B subtilis biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for stabilizing a root-associated B subtilis biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for stabilizing a root-associated B subtilis biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for stabilizing a root-associated B subtilis biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inhibiting dispersal of a root-associated bacterial biofilm comprising administering to the plant an effective amount of a composition comprising a plant-produced stimulatory compound. In another embodiment, the present invention provides a method for inhibiting dispersal of a root-associated bacterial biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for inhibiting dispersal of a root-associated bacterial biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inhibiting dispersal of a root-associated bacterial biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inhibiting dispersal of a root-associated bacterial biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

In another embodiment, the present invention provides a method for inhibiting dispersal of a root-associated B subtilis biofilm comprising administering to the plant an effective amount of a composition comprising a plant-produced stimulatory compound. In another embodiment, the present invention provides a method for inhibiting dispersal of a root-associated B subtilis biofilm comprising administering to the plant an effective amount of a composition comprising a hydroxycinnamic acid, a polyamine, a hydroxycinnamic acid-polyamine conjugate, or any combination thereof. In another embodiment, the present invention provides a method for inhibiting dispersal of a root-associated B subtilis biofilm comprising administering to the plant an effective amount of a composition comprising an analogue or derivative of hydroxycinnamic acid, polyamine, hydroxycinnamic acid-polyamine conjugate, or any combination thereof. In another embodiment, the present invention provides a method for inhibiting dispersal of a root-associated B subtilis biofilm comprising administering to the plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method for inhibiting dispersal of a root-associated B subtilis biofilm comprising administering to the plant an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof.

Without wishing to be bound by any particular theory, plant-produced stimulatory compounds activate anti-microbial agent production in certain bacterial species colonizing roots. In B. subtilis, a plant metabolite caffeic acid, 7-galloylcatechin, aesculetin, chlorogenic acid, and root exudate activatespks operon (Examples 3 and 15), which encodes an enzymatic complex responsible for synthesis of bacillaene and dihydrobacillaene, compounds which belong to class polyketides. Bacillaene and dihydrobacillaene are secreted by B. subtilis biofilms and act as antibiotics, suppressing the growth of competing bacterial species (Example 2), including pathogenic species.

Accordingly, in one embodiment, the present invention provides a method of enhancing the production of an anti-microbial agent in bacteria, the method comprising administering to said bacteria a composition comprising a hydroxycinnamic acid, a polyamine, a hydroxycinnamic acid-polyamine conjugate, or any combination thereof. In another embodiment, the present invention provides a method of enhancing the production of an anti-microbial agent in bacteria, the method comprising administering to said bacteria a composition comprising an analogue or derivative of hydroxycinnamic acid, polyamine, hydroxycinnamic acid-polyamine conjugate, or any combination thereof. In another embodiment, the present invention provides a method of enhancing the production of an anti-microbial agent in bacteria, the method comprising administering to said bacteria a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of enhancing the production of an anti-microbial agent in bacteria, the method comprising administering to said bacteria a composition comprising an analogue or derivative of caffeic acid, putrescine, caffeoyl putrescine, 7-galloylcatechin, aesculetin, chlorogenic acid, or any combination thereof.

In another embodiment, the present invention provides a method of synthesizing an anti-microbial agent in bacteria, the method comprising administering to said bacteria a composition comprising a hydroxycinnamic acid, a polyamine, a hydroxycinnamic acid-polyamine conjugate, or any combination thereof. In another embodiment, the present invention provides a method of synthesizing an anti-microbial agent in bacteria, the method comprising administering to said bacteria a composition comprising an analogue or derivative of hydroxycinnamic acid, polyamine, hydroxycinnamic acid-polyamine conjugate, or any combination thereof. In another embodiment, the present invention provides a method of synthesizing an anti-microbial agent in bacteria, the method comprising administering to said bacteria a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of synthesizing an anti-microbial agent in bacteria, the method comprising administering to said bacteria a composition comprising an analogue or derivative of caffeic acid, putrescine, caffeoyl putrescine, 7-galloylcatechin, aesculetin, chlorogenic acid, or any combination thereof.

In another embodiment, the present invention provides a method of enhancing extracellular matrix production in bacteria, the method comprising administering to said bacteria a composition comprising caffeic acid, putrescine, a caffeic acid-putrescine conjugate, 7-galloylcatechin, aesculetin, chlorogenic acid, analogue thereof, derivative thereof, or any combination thereof.

In another embodiment, the present invention provides a method of enhancing extracellular matrix production in bacteria, the method comprising administering to said bacteria a composition comprising a hydroxycinnamic acid, a polyamine, a hydroxycinnamic acid-polyamine conjugate, or any combination thereof. In another embodiment, the present invention provides a method of enhancing extracellular matrix production in bacteria, the method comprising administering to said bacteria a composition comprising an analogue or derivative of hydroxycinnamic acid, polyamine, hydroxycinnamic acid-polyamine conjugate, or any combination thereof. In another embodiment, the present invention provides a method of enhancing extracellular matrix production in bacteria, the method comprising administering to said bacteria a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of enhancing extracellular matrix production in bacteria, the method comprising administering to said bacteria a composition comprising an analogue or derivative of caffeic acid, putrescine, caffeoyl putrescine, 7-galloylcatechin, aesculetin, chlorogenic acid, or any combination thereof.

In another embodiment, the present invention provides a method of activating the pks gene operon in bacteria having a pks gene operon, the method comprising administering to said bacteria a composition comprising a hydroxycinnamic acid, a polyamine, a hydroxycinnamic acid-polyamine conjugate, or any combination thereof. In another embodiment, the present invention provides a method of activating the pks gene operon in bacteria having apks gene operon, the method comprising administering to said bacteria a composition comprising an analogue or derivative of hydroxycinnamic acid, polyamine, hydroxycinnamic acid-polyamine conjugate, or any combination thereof. In another embodiment, the present invention provides a method of activating the pks gene operon in bacteria having apks gene operon, the method comprising administering to said bacteria a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of activating the pks gene operon in bacteria having a pks gene operon, the method comprising administering to said bacteria a composition comprising an analogue or derivative of caffeic acid, putrescine, caffeoyl putrescine, 7-galloylcatechin, aesculetin, chlorogenic acid, or any combination thereof.

In another embodiment, the present invention provides a method of stimulating anti-microbial agent production by a biofilm forming bacteria colonizing roots of a plant. In another embodiment, the present invention provides a method of stimulating anti-microbial agent production by bacteria of Bacillus genus colonizing roots of a plant. In another embodiment, the present invention provides a method of stimulating polyketide antibiotic production by bacteria of Bacillus genus colonizing roots of a plant. In another embodiment, the present invention provides a method of stimulating polyketide antibiotic production by B. subtilis colonizing roots of a plant. In another embodiment, the present invention provides a method of stimulating production of bacillaene, dihydrobacillaene, or both by B. subtilis colonizing roots of a plant. In another embodiment, the present invention provides a method of stimulating production of bacillaene, dihydrobacillaene, or both by B. subtilis colonizing roots of a plant through activating B. subtilis pks operon. In another embodiment, the present invention provides a method of stimulating production of bacillaene, dihydrobacillaene, or both by Bacillus subtilis colonizing roots of a plant comprising treating B. subtilis host plant with an effective amount of a compound that activates pks operon.

In another embodiment, the present invention provides a method of stimulating production of bacillaene, dihydrobacillaene, or both by Bacillus subtilis colonizing roots of a plant comprising treating B. subtilis host plant with an effective amount of a composition comprising a hydroxycinnamic acid, a polyamine, a hydroxycinnamic acid-polyamine conjugate, or any combination thereof. In another embodiment, the present invention provides a method of stimulating production of bacillaene, dihydrobacillaene, or both by Bacillus subtilis colonizing roots of a plant comprising treating B. subtilis host plant with an effective amount of a composition comprising an analogue or derivative of hydroxycinnamic acid, polyamine, hydroxycinnamic acid-polyamine conjugate, or any combination thereof. In another embodiment, the present invention provides a method of stimulating production of bacillaene, dihydrobacillaene, or both by Bacillus subtilis colonizing roots of a plant comprising treating B. subtilis host plant with an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of stimulating production of bacillaene, dihydrobacillaene, or both by Bacillus subtilis colonizing roots of a plant comprising treating B. subtilis host plant with an effective amount of a composition comprising an analogue or derivative of caffeic acid, putrescine, caffeoyl putrescine, 7-galloylcatechin, aesculetin, chlorogenic acid, a compound according to the structures of any one of Formulas VII-XIII, or any combination thereof in order to activate pks operon.

In another embodiment, the present invention provides a method of stimulating production of bacillaene, dihydrobacillaene, or both by Bacillus subtilis colonizing roots of a plant comprising treating B. subtilis host plant with an effective amount of a composition comprising caffeic acid, 7-galloylcatechin, aesculetin, or chlorogenic acid in order to activate pks operon.

In another embodiment, the present invention provides a method of activating pks operon in B. subtilis colonizing roots of a plant comprising administering to the host plant an effective amount of a composition comprising a hydroxycinnamic acid, a polyamine, a hydroxycinnamic acid-polyamine conjugate, or any combination thereof. In another embodiment, the present invention provides a method of activating pks operon in B. subtilis colonizing roots of a plant comprising administering to the host plant an effective amount of a composition comprising an analogue or derivative of hydroxycinnamic acid, polyamine, hydroxycinnamic acid-polyamine conjugate, or any combination thereof. In another embodiment, the present invention provides a method of activating pks operon in B. subtilis colonizing roots of a plant comprising administering to the host plant an effective amount of a composition comprising caffeic acid, putrescine, caffeoyl putrescine, 7-galloylcatechin, aesculetin, chlorogenic acid, a compound according to the structures of any one of Formulas I-XIII, or any combination thereof. In another embodiment, the present invention provides a method of activating pks operon in B. subtilis colonizing roots of a plant comprising administering to the host plant an effective amount of a composition comprising an analogue or derivative ofcaffeic acid, putrescine, caffeoyl putrescine, 7-galloylcatechin, aesculetin, chlorogenic acid, a compound according to the structures of any one of Formulas VII-XIII, or any combination thereof. In another embodiment, the present invention provides a method of activating pks operon in B. subtilis colonizing roots of a plant comprising administering to the host plant an effective amount of a composition comprising caffeic acid, 7-galloylcatechin, aesculetin, or chlorogenic acid, or any combination thereof.

Activating production of polyketide antibiotics, such as bacillaene and dihydrobacillaene is effective in suppressing or preventing root colonization by many bacterial species that would otherwise compete with B. subtilis. The bacterial species susceptible to bacillaene and dihydrobacillaene include, without limitation, Klebsiella spp, Streptomyces, Pseudomonas spp, Serratia spp, Staphylococcus spp, Proteus spp, and Escherichia spp, to name a few. In one embodiment, the competing bacterial species contemplated by the present invention include S. plymuthica, and P. chlororaphis. In another embodiment, activating production of polyketide antibiotics inhibits growth or colonization of bacterial species. In one embodiment, the bacterial species comprise Klebsiella spp (e.g., Klebsiella pneumoniae), Streptomyces, Pseudomonas spp, Serratia spp (e.g. Serratia marcescens), Proteus spp (e.g., Proteus vulgaris), Staphylococcus spp (e.g., Staphylococcus aureus), Bacillus thuringiensis, and Escherichia spp (e.g. Escherichia coli).

In addition, the methods of the present invention provide activation of a pks gene operon, in one embodiment, in a bacterial species having a pks gene operon. In one embodiment, the bacteria comprise Bacillus. In another embodiment, the bacteria comprise Streptomyces, which in one embodiment, comprises Streptomyces atroolivaceus. In another embodiment, the bacteria comprise Pseudomonas, which in one embodiment, comprises Pseudomonas fluorescens. In another embodiment, the bacteria comprise Myxococcus, which in one embodiment, comprises Myxococcus xanthus. In another embodiment, the bacteria comprise Amycolatopsis, which in one embodiment, comprises Amycolatopsis mediterranei.

In one embodiment, the anti-microbial agent expressed by the bacteria biofilm comprises an anti-bacterial agent. In one embodiment, the anti-bacterial agent comprises a polyketide antibiotic. In one embodiment, the polyketide antibiotic comprises bacillaene, dihydrobacillaene, or a combination thereof. In another embodiment, the anti-microbial agent expressed by the bacteria biofilm comprises an anti-fungal agent.

In one embodiment, the bacteria used in the compositions and methods as described herein are B. subtilis. In one embodiment, the bacteria for the compositions and methods as described herein are present in a biofilm. In one embodiment, the bacteria for the compositions and methods as described herein are present on a root of a plant. In another embodiment, the bacteria for the compositions and methods as described herein are present in an in vitro culture. In one embodiment, the composition as described herein is directly administered to bacteria present on or near the plant. In another embodiment, the composition as described herein is directly administered to bacteria in culture. In another embodiment, the composition as described herein is administered to a plant, wherein bacteria are present on an organ of the plant.

Transgenic Plants

The present invention further provides a transgenic plant or a genetically modified plant, having enhanced production of caffeic acid, putrescine, caffeoyl putrescine, 7-galloylcatechin, aesculetin, chlorogenic acid, or any combination thereof. In another embodiment, the present invention provides a transgenic plant or a genetically modified plant, having enhanced production of a hydroxycinnamic acid, a polyamine, a hydroxycinnamic acid-polyamine conjugate, or any combination thereof. In another embodiment, the present invention provides a transgenic plant or a genetically modified plant, having enhanced production of an analogue or derivative of caffeic acid, putrescine, caffeoyl putrescine, a hydroxycinnamic acid, a polyamine, a hydroxycinnamic acid-polyamine conjugate, 7-galloylcatechin, aesculetin, or chlorogenic acid, or any combination thereof.

The present invention further provides a transgenic plant having one or more genetically modified cells that exhibit enhanced synthesis of caffeic acid, putrescine, caffeoyl putrescine, 7-galloylcatechin, aesculetin, chlorogenic acid, or any combination thereof; a hydroxycinnamic acid, a polyamine, a hydroxycinnamic acid-polyamine conjugate, or any combination thereof; an analogue or derivative of caffeic acid, putrescine, caffeoyl putrescine, a hydroxycinnamic acid, a polyamine, a hydroxycinnamic acid-polyamine conjugate or any combination thereof, 7-galloylcatechin, aesculetin, or chlorogenic acid, when compared to corresponding plant cells that have not been genetically modified.

Methods of producing transgenic plants are well known to those of ordinary skill in the art. Transgenic plants can be produced by a variety of different transformation methods including, but not limited to, electroporation; microinjection; microprojectile bombardment, also known as particle acceleration or biolistic bombardment; viral-mediated transformation; Agrobacterium-mediated transformation, and water-soluble fullerene derivatives-mediated transformation (see, e.g., U.S. Pat. Nos. 5,405,765, 5,472,869, 5,538,877, 5,538,880, 5,550,318, 5,641,664, 5,736,369, 6,255,559, and 8,614,366; Watson et al., Recombinant DNA, Scientific American Books (1992); Hinchee et al., Bio/Tech 6:915-922 (1988); McCabe et al., Bio/Tech. 6:923-926 (1988); Toriyama et al., Bio/Tech 6:1072-1074 (1988); Fromm et al., Bio/Tech. 8:833-839 (1990); Mullins et al., Bio/Tech. 8:833-839 (1990); and, Raineri et al., Bio/Tech. 8:33-38 (1990), all of which are hereby incorporated by reference in their entirety).

In one embodiment, all the cells of the genetically modified plants of this invention are genetically modified. In another embodiment, a subset of the cells of the genetically modified plants of this invention is genetically modified. In one embodiment, the genetically modified cells are distributed throughout the plant. In another embodiment, the genetically modified cells are concentrated in a particular part of a plant, e.g., stem, flowers, leaves, branches, seeds, or roots. Each possibility represents a separate embodiment of invention.

In one embodiment, the genetically modified cells are concentrated in one or more roots of a plant.

In one embodiment, the present invention provides methods of increasing the amount of caffeic acid, putrescine, caffeoyl putrescine, 7-galloylcatechin, aesculetin, chlorogenic acid, or any combination thereof; a hydroxycinnamic acid, a polyamine, a hydroxycinnamic acid-polyamine conjugate, or any combination thereof, an analogue or derivative of caffeic acid, putrescine, caffeoyl putrescine, 7-galloylcatechin, aesculetin, or chlorogenic acid, a hydroxycinnamic acid, a polyamine, a hydroxycinnamic acid-polyamine conjugate, or any combination thereof from a plant that is released from the root of a plant.

In one embodiment, increasing the amount of released compounds comprises enhancing the release of the compound. In another embodiment, increasing the amount of released compounds comprises increasing the synthesis of the compounds in the plant. In another embodiment, increasing the amount of released compounds comprises decreasing the breakdown of the compounds in the plant. In another embodiment, increasing the amount of released compounds comprises providing environmental conditions that increase production and/or release of the compounds. In one embodiment, the environmental condition that increases production and/or release of the compound comprises low iron levels in the environment of the plant.

In one embodiment, increasing the amount of released compounds comprises administering to the plant a composition comprising enzymes from the sam1-sam8 enzyme cluster from E. coli. In another embodiment, increasing the amount of released compounds comprises administering to the plant a composition comprising phenylalanine ammonia lyase (PAL), trans-cinnamate 4-hydroxylase (C4H), p-coumarate 3-hydroxylase (C3H), or a combination thereof.

In another embodiment, increasing the amount of released compounds comprises administering to the plant a composition comprising a bioactive polypeptide, such as an enzyme, that increases the synthesis and/or release of caffeic acid, 7-galloylcatechin, aesculetin, or chlorogenic acid. In another embodiment, increasing the amount of released compounds comprises administering to the plant a composition comprising a polynucleotide encoding a polypeptide that increases the synthesis and/or release of caffeic acid, 7-galloylcatechin, aesculetin, chlorogenic acid, or any combination thereof.

Methods of generating transgenic plant cells having enhanced caffeic acid, 7-galloylcatechin, aesculetin, or chlorogenic acid synthesis will be well known to those of ordinary skill in the art and, in one embodiment, include introducing to plant cells a transgene encoding enzymes responsible for caffeic acid synthesis. In one embodiment, an enzyme involved in caffeic acid synthesis comprises the sam1-sam8 enzyme cluster from E. coli (Berner et al. J Bacteriol. 188(7): 2666-2673. (2006), incorporated by reference in their entirety). In another embodiment, an enzyme involved in caffeic acid biosynthesis in plants comprises phenylalanine ammonia lyase (PAL), trans-cinnamate 4-hydroxylase (C4H), p-coumarate 3-hydroxylase (C3H) (U.S. Pat. No. 8,809,028, incorporated by reference in their entirety).

Additionally, methods of limiting transgene synthesis to a particular section of the plant (e.g. roots) will be known to a skilled artisan. One approach is, for example, to use root specific promoters to drive transgene expression. Such root specific promoters have been described in the art (see e.g. Dutt et al., Horticulture Research 1: 14047 (2014), incorporated herein by reference in its entirety).

Plants

The methods of present invention are applicable to a wide variety of cultivated plants. As used herein, the term “plant” refers to any living organism belonging to the kingdom Plantae (i.e., any genus/species in the Plant Kingdom). This includes familiar organisms such as, but not limited to, trees, herbs, bushes, grasses, vines, ferns, mosses, and green algae. The term refers to both monocotyledonous plants, also called monocots, and dicotyledonous plants, also called dicots. Examples of particular plants include, but are not limited, to apple trees, citrus fruits (e.g. grapefruit, lemon, lime, orange, tangerine, citrus hybrids, pummelo, and other citrus fruit crops), stone fruits (e.g., coffees, jujubes, mangos, olives, coconuts, oil palms, pistachios, almonds, apricots, cherries, damsons, nectarines, peaches and plums), and the like. For a more complete list of representative crop plants see, for example, Glossary of Crop Science Terms: Ill, Nomenclature, Common and Scientific Names, Crop Science Society of America, July 1992, which is herein incorporated in its entirety. In one embodiment, the plant comprises a decorative plant. In another embodiment, the plant comprises a crop plant.

In one embodiment, the plant comprises a dicot plant. In another embodiment, the plant comprises a member of the family Amaranthaceae, for example, a spinach plant, or a quinoa plant. In another embodiment, the plant comprises a member of the family Anacardiaceae, for example, a mango plant. In another embodiment, the plant comprises a member of the family Asteraceae, for example, a sunflower plant, an endive plant, a lettuce plant, or an artichoke plant. In another embodiment, the plant comprises a member of the family Bromeliaceae, for example, a pineapple plant. In another embodiment, the plant comprises a member of the family Caricaceae, for example, a papaya plant. In another embodiment, the plant comprises a member of the family Chenopodiaceae, for example, a beet plant. In another embodiment, the plant comprises a member of the family Curcurbitaceae, for example, a melon plant, a cantaloupe plant, a squash plant, a watermelon plant, a honeydew plant, a cucumber plant, or a pumpkin plant. In another embodiment, the plant comprises a member of the family Dioscoreaceae, for example, a yam plant. In another embodiment, the plant comprises a member of the family Ericaceae, for example, a blueberry plant. In another embodiment, the plant comprises a member of the family Euphorbiaceae, for example, a cassava plant. In another embodiment, the plant comprises a member of the family Fabaceae, for example, an alfalfa plant, a clover plant, or a peanut plant. In another embodiment, the plant comprises a member of the family Grossulariaceae, for example, a currant plant. In another embodiment, the plant comprises a member of the family Juglandaceae, for example, a walnut plant. In another embodiment, the plant comprises a member of the family Lamiaceae, for example, a mint plant. In another embodiment, the plant comprises a member of the family Lauraceae, for example, an avocado plant. In another embodiment, the plant comprises a member of the family Leguminosae, for example, a soybean plant, a bean plant, or a pea plant. In another embodiment, the plant comprises a member of the family Malvaceae, for example, a cotton plant. In another embodiment, the plant comprises a member of the family Marantaceae, for example, an arrowroot plant. In another embodiment, the plant comprises a member of the family Myrtaceae, for example, a guava plant, or a eucalyptus plant. In another embodiment, the plant comprises a member of the family Rosaceae, for example, a peach plant, an apple plant, a cherry plant, a plum plant, a pear plant, a prune plant, a blackberry plant, a raspberry plant, or a strawberry plant. In another embodiment, the plant comprises a member of the family Rubiaceae, for example, a coffee plant. In another embodiment, the plant comprises a member of the family Rutaceae, for example, a citrus plant, an orange plant, a lemon plant, a grapefruit plant, or a tangerine plant. In another embodiment, the plant comprises a member of the family Salicaceae, for example, a poplar plant, or a willow plant. In another embodiment, the plant comprises a member of the family Solanaceae, for example, a potato plant, a sweet potato plant, a tomato plant, a Capsicum plant, a tobacco plant, a tomatillo plant, an eggplant plant, an Atropa belladonna plant, or a Datura stramonium plant. In another embodiment, the plant comprises a member of the family Triticeae, for example, a wheat plant, a barley plant, a rye plant, a triticale plant, or an oats plant. In another embodiment, the plant comprises a member of the family Vitaceae, for example, a grape plant. In another embodiment, the plant comprises a member of the family Umbelliferae, for example, a carrot plant. In another embodiment, the plant comprises a member of the family Musaceae, for example, a banana plant. In another embodiment the plant comprises any cultivated plant.

In one embodiment, the plant comprises a member of the family Brassicaceae, which includes, without limitation, Arabidopsis thaliana, Brassica oleracea (cabbage, broccoli, Brussels sprouts, kohlrabi, or cauliflower), Brassica rapa (turnip), Brassica napus (rapeseed), mustard, Raphanus raphanistrum (radish), Armoracia rusticana (horseradish), Lepidium sativum (cress), Eutrema japonicum (wasabi), Eruca sativa (arugula), Nasturtium officinale (watercress), Brassica juncea, (brown mustard), or canola. Each possibility represents a separate embodiment of invention.

In one embodiment, the plant comprises a Solanum lycopersicum (tomato), Solanum tuberosum (potato), Zea mays (corn), or Triticum aestivum (wheat). Each possibility represents a separate embodiment of invention.

Diseases

The methods of present invention are can be used to treat a wide variety of plant diseases. As used herein, the term “disease”, as applied to a plant, refers to an impairment in the structure or function of a plant that results in observable symptoms. In one embodiment a plant disease can be caused by an infectious organism (“pathogen”). In another embodiment, a plant disease can be caused by an environmental situation that isn't suited for or doesn't agree with the plant. In a further embodiment, a plant disease can be caused by a combination of pathogens and environmental factors. As used herein, the term “treat” means to decrease, suppress, attenuate, diminish, arrest, stabilize or prevent the development or progression of a disease or disorder delineated herein, lessen the severity of the disease or disorder, or improve the symptoms associated with the disease or disorder.

Exemplary disease or conditions include, but are not limited to bacterial blight, brown spot, common blight, vascular wilt, white mold, root rots, head blight, fire blight, silver scurf, dry rot, common scab, ring rot, soft rot, damping off, seedling blight, seed rot, and bacterial canker. Each possibility represents a separate embodiment of invention.

In one embodiment, the plant disease comprises a bacterial infestation or infection. The bacterial infections contemplated in the present invention include, without limitation, diseases or conditions caused by Pseudomonas spp., Xanthomonas spp., Curtobacterium spp., Sclerotinia spp., Pythium spp., Fusarium spp., Botrytis cinerea, Helminthosporium solani, Streptomyces spp., Phytophthora spp., Rhizoctonia solani, Erwinia pp., and Clavibacter spp., to name just a few. Each possibility represents a separate embodiment of invention. In one embodiment, the bacterial infestation or infection susceptible to treatment by the methods of the present invention comprises Pseudomonas syringae infestation or infection or Xanthomonas campestris infestation or infection.

In one embodiment, the plant disease comprises a fungal infestation or infection. The fungal infections contemplated in the present invention include, without limitation, diseases or conditions caused by Alternaria spp., Ascochyta spp., Botrytis cinerea, Cercospora spp., Claviceps purpurea, Cochliobolus sativus, Colletotrichum spp., Epicoccum spp., Fusarium graminearum, Fusarium moniliforme, Fusarium oxysporum, Fusarium proliferatum, Fusarium solani, Fusarium subglutinans, Gaumannomyces graminis, Helminthosporium spp., Microdochium nivale, Penicillium spp., Phoma spp., Pyrenophora graminea, Pyricularia oryzae, Rhizoctonia solani, Rhizoctonia cerealis, Sclerotinia spp., Septoria spp., Sphacelotheca reilliana, Tilletia spp., Typhula incarnata, Urocystis occulta, Ustilago spp. or Verticillium spp., to name just a few. Each possibility represents a separate embodiment of invention. In one embodiment, the bacterial infestation or infection susceptible to treatment by the methods of the present invention comprises Rhizoctonia solani infestation or infection or Fusarium graminearum infestation or infection.

Methods of Isolating Caffeoyl Putrescine

In one embodiment, the present invention provides a method for isolating caffeoyl putrescine from the root or root exudate of a plant, the method comprising: (a) obtaining root exudate from said plant; (b) fractionating and purifying said exudate; (c) dansylating amine groups in the fraction of said exudate that blocks B. subtilis biofilm dispersal; and (d) resolving dansylated compounds from selected fractions using thin layer chromatography, thereby isolating caffeoyl putrescine from the root or root exudate of said plant.

The present invention further provides a method for isolating Root Inhibitor of Dispersal (RID) compounds from root exudate of a plant. In one embodiment, RID is isolated through a procedure comprising obtaining a root exudate of a plant using methods described herein, fractionating said exudates using methods well known in the art (e.g. ion exchange chromatography), chemically derivatizing compounds in each fraction with dansyl chloride, testing dansylated compounds in each fraction for ability to block B. subtilis biofilm dispersal using methods described herein (e.g. pellicle development assay), selecting the fractions that block B. subtilis biofilm dispersal and resolving individual dansylated compounds from selected fractions using well known methods (e.g. thin layer chromatography), thereby identifying RID compounds. In one embodiment, RID is a purified fraction of a plant root exudate. In one embodiment, RID comprises caffeoyl putrescine.

In one embodiment, the fractionation step of the method comprises HPLC chromatography. In another embodiment, the fractionation step of the inventive method comprises Liquid chromatography-mass spectrometry. In another embodiment, the fractionation step of the inventive method comprises any chromatographic fractionation method known in the art. In another embodiment, the fractionation step of the inventive method comprises several chromatographic steps. In a preferred embodiment, the fractionation step of the inventive method comprises HPLC chromatography, followed by Liquid chromatography-mass spectrometry (LC-MS). It is well within competence of a skilled artisan to select the methods of fractionation from among those known in the art, and to optimize the order of the steps of fractionation.

In one embodiment, in the fraction capable of blocking B. subtilis biofilm dispersal is tested in step (c) with a pellicle development assay. In one embodiment, the dansylation in step (c) comprises derivatization of amine groups with dansyl chloride.

In one embodiment, the plant was optionally grown under iron-deficient conditions.

In another embodiment, the present invention provides a method of transiently enhancing caffeic acid production in plants using transient transformation of plants. Transient transformation of plants with DNA and RNA expression vectors has been described in the art (see e.g. Guidarelli and Baraldi, Front Plant Sci 6: 444 (2015), incorporated by reference in their entirety) and can be used to deliver DNA and RNA transient expression vectors encoding enzymes involved in caffeic acid, 7-galloylcatechin, aesculetin, or chlorogenic acid biosynthesis in plants. The present invention further contemplates transiently enhancing caffeic acid production through direct delivery of enzymes involved in caffeic acid, 7-galloylcatechin, aesculetin, or chlorogenic acid biosynthesis in plants into cells of a plant. The methods of intracellular delivery of proteins in plants (e.g. through use of fusion peptides) have been described in the art (see Ng et al. PLoS One, 11(4): e0154081 (2016), incorporated by reference in their entirety).

Methods of Administration

Persons of skill are aware of various methods to apply compounds, to plants for surface application or for uptake, and any of these methods are contemplated for use in this invention. Methods of administration to plants include, by way of non-limiting example, application to any part of the plant, by inclusion in irrigation water, by injection to the plant or to the soil surrounding the plant, or by exposure of the root system to aqueous solutions containing the compounds, by use in hydroponic or aeroponic systems, by seed treatment, by exposure of cuttings of plants used for grafting to aqueous solutions containing the compounds, by application to the roots, stems or leaves, by application to the plant interior, or any part of the plant to be treated. Any means known to those of skill in the art is contemplated.

Suitable amounts for administration to a plant are in the range of about 20 mL to about 1000 mL for trunk injection, the range of about 0.1 gallons per plant to about 0.8 gallons per plant for foliar spraying, and the range of about 0.25 gallons per to about 2 gallons per plant for soil drench and soil injection methods. Therefore, for trunk injection, amounts to be administered to a plant are about 20 mL to about 1000 mL, preferably about 100 mL to about 800 mL, and most preferably about 300 mL to about 600 mL. For foliar spraying, amounts to be administered to a plant are about 0.1 gallons per plant to about 0.8 gallons per plant, preferably about 0.2 gallons per plant to about 0.6 gallons per plant, and most preferably about 0.25 gallons per plant to about 0.5 gallons per plant. For soil drench and soil injection, amounts to be administered to a plant are about 0.25 gallons per plant to about 2 gallons per plant, preferably about 0.3 gallons per plant to about 1.5 gallons per plant, and most preferably about 0.5 gallons per plant to about 1 gallon per plant. Persons of skill in the art are able to adjust these amounts taking into account the plant size, timing of application and environmental conditions.

Application of the compounds can be performed in a nursery setting, a greenhouse, hydroponics facility, or in the field, or any setting where it is desirable to treat plants which have been or can become exposed to a plant disease, such as S. plymuthica or P. chlororaphis infection, or which can benefit from fitness improvement. The methods and compositions of this invention can be used to treat infection with a plant pathogen and can be used to improve plant defenses or health, growth and productivity in plants which are not infected. Thus, any plant in need, in the context of this invention, includes any plant susceptible to a lack of optimum fitness, or susceptible to a plant disease, whether currently infected or in potential danger of infection, in the judgement of the person of skill in this and related arts.

Application to soil is preferably performed by soil injection or soil drenching, however any method known in the art can be used. These methods of administration are accomplished as follows. Soil drenching may be performed by pouring a solution or vehicle containing caffeic acid, putrescine, a caffeic acid-putrescine conjugate, 7-galloylcatechin, aesculetin, or chlorogenic acid, or any combination thereof to the soil surface, optionally by using the irrigation system. Soil injection may be performed by directly injecting a solution or vehicle containing caffeic acid, putrescine, a caffeic acid-putrescine conjugate, 7-galloylcatechin, aesculetin, or chlorogenic acid, or any combination thereof into the soil using a soil injector. The injection area relative to a plant, concentrations, volumes, and duration may change depending on the plant and can be determined by one of skill in the art. However, preferred methods are those wherein the administering to the plant provides the local concentration of at about 1-150 μM caffeic acid, putrescine, a caffeic acid-putrescine conjugate, 7-galloylcatechin, aesculetin, or chlorogenic acid, or any combination thereof in the B. subtilis biofilm at the roots of the plant.

Application to hydroponic or culture media preferably is performed as follows, however any method known in the art can be used. A solution or vehicle containing caffeic acid, putrescine, a caffeic acid-putrescine conjugate, 7-galloylcatechin, aesculetin, or chlorogenic acid, or any combination thereof of 1-150 μM may be added into the hydroponic or culture media at final concentrations of 1-150 μM. The concentrations and volumes may change depending on the plant, and can be determined by one of skill in the art.

Application to the roots preferably is performed by immersing the root structure in a solution or vehicle in a laboratory, nursery or hydroponics environment, or by soil injection or soil drenching to the soil surrounding the roots, as described above. Emersion of the root structure preferably is performed as follows, however any method known in the art can be used. A solution or vehicle containing caffeic acid, putrescine, a caffeic acid-putrescine conjugate, 7-galloylcatechin, aesculetin, or chlorogenic acid, or any combination thereof may be applied to the roots by using a root feeder. The concentrations, volumes, and duration may change depending on the plant and can be determined by one of skill in the art, however preferred methods are those wherein the administering to the plant provides the local concentration of at about 1-150 μM caffeic acid, putrescine, a caffeic acid-putrescine conjugate, 7-galloylcatechin, aesculetin, or chlorogenic acid, or any combination thereof in the B. subtilis biofilm at the roots of the plant.

Application to the stems or leaves ofthe plant preferably is performed by spraying or other direct application to the desired area of the plant, however any method known in the art can be used. A solution or vehicle containing caffeic acid, putrescine, a caffeic acid-putrescine conjugate, 7-galloylcatechin, aesculetin, or chlorogenic acid, or any combination thereof may be applied with a sprayer to the stems or leaves until runoff to ensure complete coverage, and repeat three or four times in a growing season. The concentrations, volumes and repeat treatments may change depending on the plant and can be determined by one of skill in the art.

Application to the plant interior preferably is performed by injection directly into the plant, for example by trunk injection or injection into a limb, however any method known in the art can be used. A solution or vehicle containing caffeic acid, putrescine, a caffeic acid-putrescine conjugate, 7-galloylcatechin, aesculetin, or chlorogenic acid, or any combination thereof may be applied with an injector into the plant interior, and repeat three or four times in a growing season. The concentrations, volumes and repeat treatments may change depending on the plant and can be determined by one of skill in the art.

Preferred methods of administration are soil application methods, including soil injection, soil soaking or soil spraying. One method according to the invention for treatment of trees comprises application to the soil by soil injection within a 10-foot radius of a plant to be treated, for example a plant exhibiting infection with or symptoms of infection with a plant pathogen. Any method of administering the bacteria which contacts the bacteria with the roots of the plant is preferred. The concentrations, volumes, and duration may change depending on the plant and can be determined by one of skill in the art, however preferred methods are those wherein the administering to the plant provides the local concentration of at about 1-150 μM caffeic acid, putrescine, a caffeic acid-putrescine conjugate, 7-galloylcatechin, aesculetin, or chlorogenic acid, or any combination thereof in the B. subtilis biofilm at the roots of the plant.

The present invention will be further illustrated in the following examples. However, it is to be understood that these examples are for illustrative purposes only, and should not be used to limit the scope of the present invention in any manner.

### EXAMPLES

### Materials and Methods for Examples 1-5

Strains and Media. The strains used in this study are listed in Table 1. All experiments were performed with B. subtilis NCBI3610 and its derivatives. 16S analysis was used to confirm the specie of the S. plymuthica and P. chlororaphis strains. Assays were carried out with the biofilm-inducing medium, MSgg: 5 mM potassium phosphate, 100 mM MOPS (pH 7), 2 mM MgCl₂, 50 μM MnCl₂, 1 μM ZnCl₂, 2 μM thiamine, 0.5% glycerol, 0.5% glutamate, 50 μg/ml tryptophan, 50 μg/ml phenylalanine 50 μg/ml threonine, 700 μM CaCl₂, and 50 μM (for growth assays) or 125 μM (for biofilm assays) FeCl₃ (Branda et al 2001, Kolodkin-Gal et al 2013). A solid MSgg medium was obtained by adding Bacto agar (Difco) to a final concentration of 1.5%. When necessary, selective medium was prepared with LB-agar or LB broth, supplemented with: 100 μg/ml ampicillin (amp) (AG Scientific), or 10 μg/ml kanamycin (kan) (AG Scientific), or 10 μg/ml chloramphenicol (cam) (Amresco), or 10 μg/ml tetracycline (tet) (Amresco), or 100 μm/ml spectinomycin (spec) (Tivan Biotech), or 1 μg/mlerythromycin (erm) (Amresco)+25 μg/ml lincomycin (linc) (Sigma Aldrich). To create the transcriptional luc reporter, the promoter of pksC was amplified from gDNA of B. subtilis NCIB 3610 with the following primers: Primer 5′: Ppks-lux P1 gtcctagtaaggtcgacaggaggactctctgcaaatcgcccggccattcgataaagg. Primer 3′: P_{pksC}-lux P2 gtatgtaagcaaaaagtttccaaatttcattctctcaaagccacccttccgattagt and further integrated into pBS3Clux by restriction free cloning. After confirmation by sequencing, pBS3Clux was cut and integrated into the bacterial genome of B. subtilis NCIB 3610 at the neutral sacA locus.

Deletion of CodY were generated using the long-flanking homology (LFH) PCR mutagenesis protocol of Wach (1996), replacing an endogenous locus with a resistance gene from plKec14 (kan). Primers used were as following: P1, TCGATATGGATGAAGTCGGCCAGGAA; P2, CAATTCGCCCTATAGTGA GTCGTCCGCAGCTTGCAGCATGGAGTTAATA; P3, CCAGCTTTTGTTCCCTT TAGTGAGTCAGGCTTATATCAAGGCGAGAAATGTAGTT; P4, TTCTGTAAGGC ACCCACTCTCCATTC

The product was first introduced by transformation into strain B. subtilis PY79 and the deletion further integrated into NCIB 3610 wild-type or mutant by transformation. Transformation of B. subtilis PY79 and NCIB 3610 by natural competence with linearized plasmid or genomic DNA was done as described (Wilson and Bott 1968) and as performed to assess natural competence in 3610 (Konkol et al 2013).

### Table 1. Strains Used in the Present Study

| Name | Genotype | reference/source |
|---|---|---|
| B. subtilis | | |
| NCIB3610 | wild-type | (Conn H. J. 1930) |
| IKbs0222 | Δhag::tet | Angelini, et al. (2009). |
| | | Proc Natl Acad Sci USA |
| | | 106, 18109-18113. |
| IKbs0210 | ΔsigD::tet, | Lab collection |
| | amyE::P_{hag}-gfp | |
| | (Cam) | |
| IKbs0225 | ΔcheA::tet | Kearns, et al. (2003). |
| | | Mol Microbiol 49, 581- |
| | | 590. |
| IKbs0226 | ΔcheY::tet | Kearns, et al. (2003). |
| | | Mol Microbiol 49, 581- |
| | | 590. |
| IKbs0008 | ΔepsH::tet | Kearns, et al. (2005). |
| | | Mol Microbiol 55, 739- |
| | | 749. |
| IKbs0005 | ΔtasA::kan | Vlamakis, et al. (2008). |
| | | Genes & development |
| | | 22, 945-953. |
| IKbs0227 | ΔsrfAA::mls | Angelini, et al. (2009). |
| | | Proc Natl Acad Sci USA |
| | | 106, 18109-18113. |
| IKbs0042 | Δvpr::kan | Lab collection |
| IKbs0041 | Δmpr::tet | Lab collection |
| IKbs0036 | ΔaprE::mls | Lab collection |
| IKbs0224 | ΔpksX::spec | Butcher, et al. (2007). |
| | | Proc Natl Acad Sci USA |
| | | 104, 1506-1509, As pksX |
| | | was found to be essential for |
| | | PKS activity. It is referred |
| | | to it as a pks null |
| | | throughout the text. |
| IKbs0035 | ΔpksA-E::tet | Straight, et al. (2007). |
| | | Proc Natl Acad Sci USA |
| | | 104, 305-310. |
| Serratia plymuthica | wild-type | Kindly Provided by |
| | | Prof. L. Chernin |
| Pseudomonas | wild-type | Kindly Provided by |
| Chlororaphis PC-449 | | Prof. L. Chernin |

Bacillaene extraction. Bacillaene was extracted from the biofilms of strain NCIB3610. A single B. subtilis WT colony, isolated on a solid LB plate, was inoculated into 3 ml of LB broth, grown overnight at room temperature and 2 μl of the culture was plated onto a solid MSgg medium. The plate was incubated for three days at 30° C. to allow proper biofilm development. A ˜44 mm² MSgg-agar segment was cut out of the plate (together with a piece of the biofilm), crushed manually and 700 μl of isopropanol was added to it. The isopropanol-crushed MSgg mixture was then incubated at 50° C. for 1 hr, with occasional vortexing, followed by centrifugation (14,000 rpm for 5 min) to precipitate MSgg-agar flakes. Supernatant was extracted and evaporated by speed-vac. As a control, the same procedure was performed with a non-inoculated solid-MSgg medium. To verify that the activity of the extract was pks-dependent and not due to extraction of surfactin, the procedure was also carried out with ΔpksA-R.

To analyze the activity of the extracted molecules, 6.6 μl DMSO and 193.4 μl dW were added to the evaporated residues and vortexed to dissolve them (resulting in a molecule concentration of 3.5×). For S. plymuthica growth curves, liquid MSgg was supplemented with 30% of the 3.5× solution, achieving a final concentration of ˜1× and growth was performed in a microplate reader, as described. Cells were then grown at room temperature with agitation for 18 hr in a microplate reader (Synergy 2, BioTek) and optical density was measured at 600 nm (OD₆₀₀) every 15 min.

Isopropanol extracts were analyzed by liquid chromatography-mass spectrometry. HPLC separations were carried out using Kinetex Hexyl-Phenyl column (2.1×150 mm, particle size 2.6 μm, Phenomenex). Mass spectrometer was operated in positive ionization mode, ion source parameters were as follows: spray voltage 3.5 kV, capillary temperature 300° C., ion-transfer optics parameters were optimized using automatic tune option, sheath gas rate (arb) 35, and auxiliary gas rate (arb) 15. Mass spectra were acquired in the m/z 150-2000 Da range. The LC-MS system was controlled and data were analyzed using Chromeleon and Xcalibur software (Thermo Fisher Scientific Inc.). Bacillaene was detected by m/z 581.3585. Dihydrobacillaene was not detected.

Interaction assays. A single B. subtilis or S. plymuthica colony, isolated on solid LB plates, was inoculated into 3 ml of LB broth (Difco) and grown overnight at room temperature. Interactions were accomplished by inoculating 2 μl of each culture onto solid MSgg mediums, at the desired distances. Plates were incubated at 23° C. and 30° C. for five and three days, respectively. Images were obtained by a stereomicroscope (Zeiss).

The calculation of the radius ratio of B. subtilis biofilm colony. The radius ratio was determined by dividing one radius, extending from the center of the biofilm towards the S. plymuthica colony, by the second radius, extending to the opposite side. This value enabled a comparison of the relative radius ratios between different B. subtilis deletion mutants, lacking factors that may have a potential involvement in the asymmetrical extension.

The effect of S. plymuthica supernatant or cells debris on B. subtilis To acquire S. plymuthica supernatant and dead cells, an overnight LB culture of S. plymuthica grown at room temperature, was inoculated into liquid MSgg medium (dilution of 1:100) and incubated at 30° C. for 24 hr. Cultures were then centrifuged (14,000 rpm, 10 min) and the supernatant was filtered, evaporated by speed-vac and rehydrated with distilled water (dW), achieving a 20× concentration. Remaining cells were washed with dW and re-suspended in 2 ml dW. Cells were heat-killed at 85° C. for 25 min. For the interaction, 3 μl of either the 20× supernatant or the dead cells were inoculated near a 2 μl B. subtilis LB culture (grown at room temperature overnight) and then incubated at 30° C. for three days.

Imaging of the interaction between biofilms. Single B. subtilis or S. plymuthica colony, isolated on solid LB plwHENates, was inoculated into 3 ml of LB broth (Difco) and grown overnight at room temperature. Interactions were accomplished by inoculating 2 μl of each culture onto solid MSgg mediums, at the desired distances. Plates were incubated at 23° C. and 30° C. for five and three days, respectively. Images were obtained by a stereomicroscope (Zeiss). Image analysis was performed with ImageJ.

Analysis of bacterial populations in the interaction. To analyze cell number of each bacterium during the interaction between B. subtilis and S. plymuthica, interaction plates were prepared, as described, and incubated for the required time period.

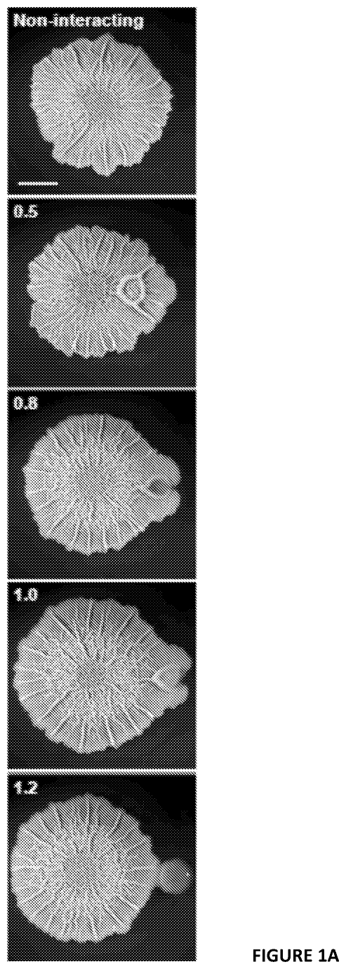

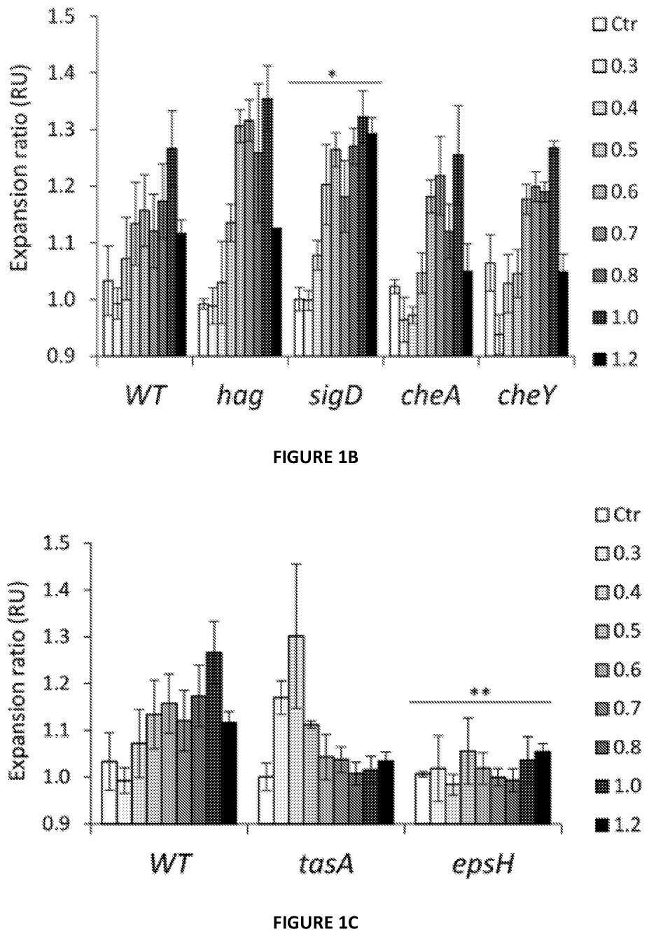

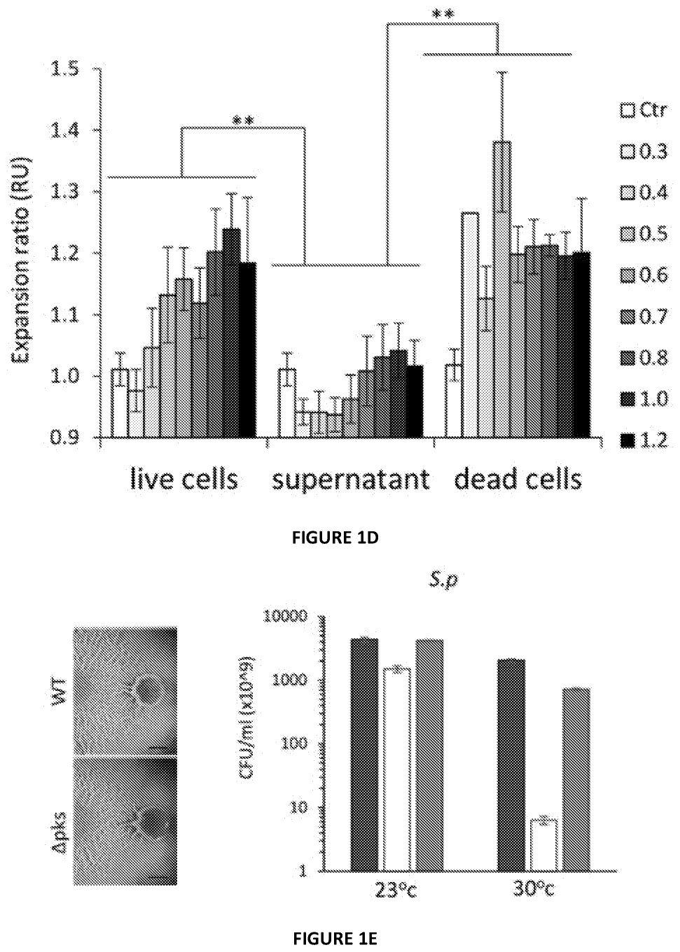

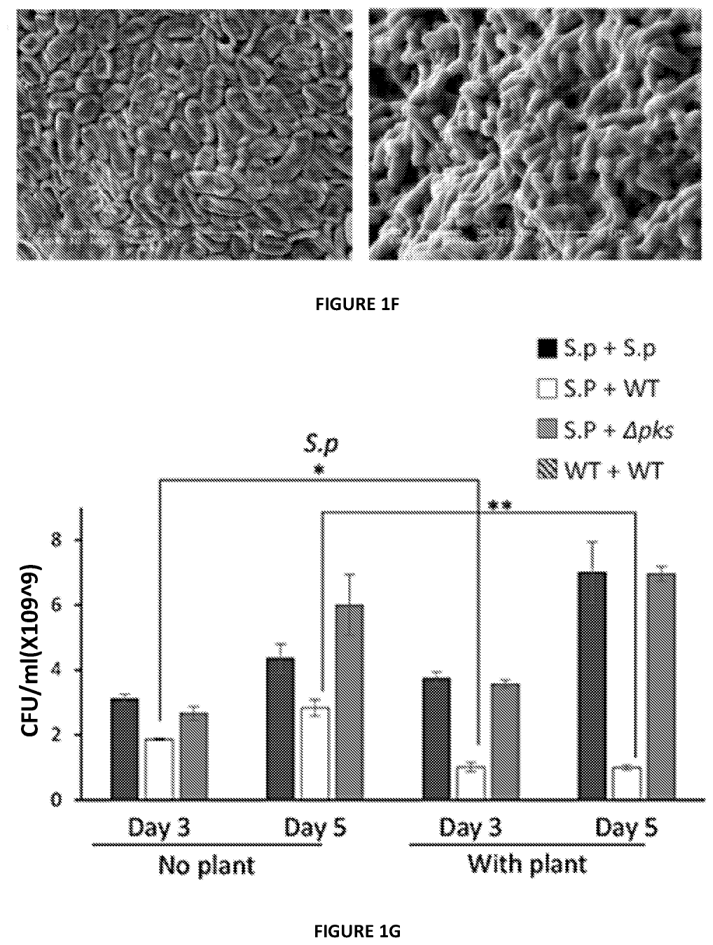

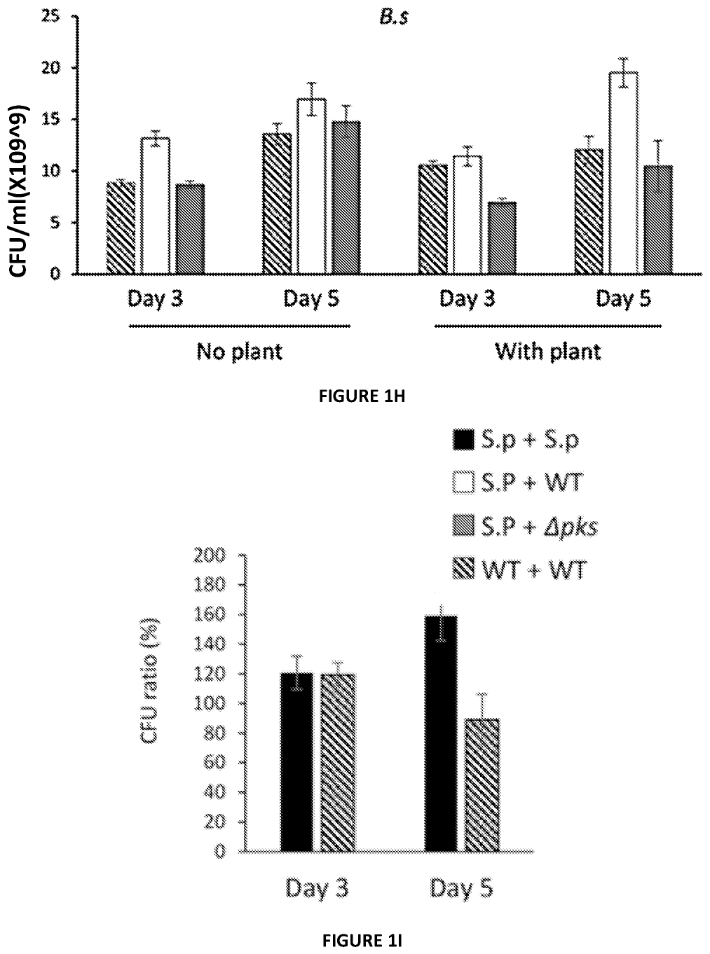

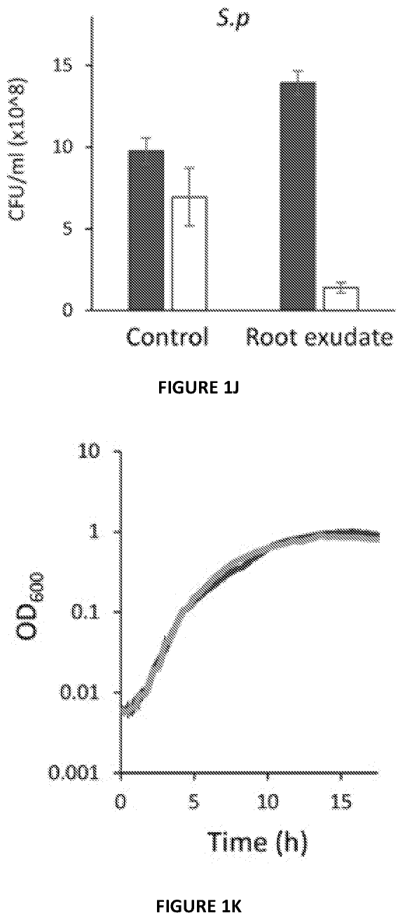

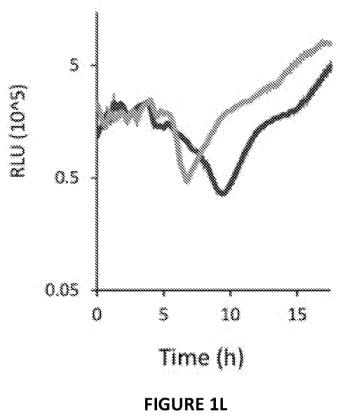

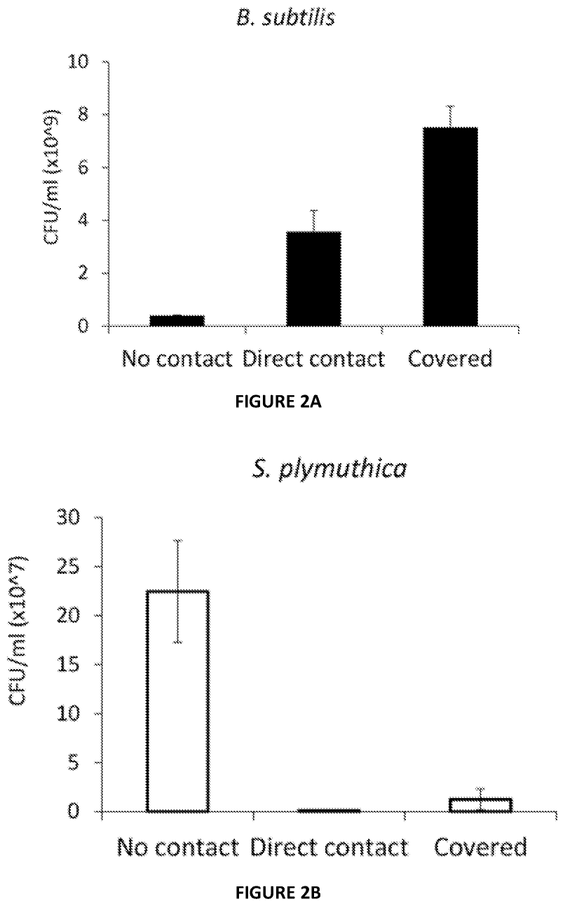

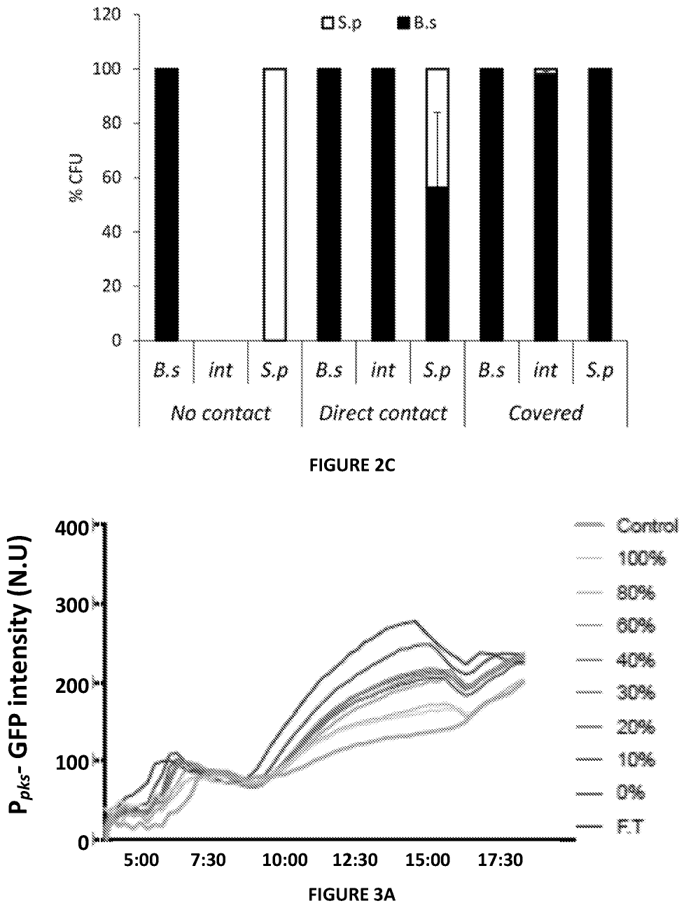

For FIGS. 1A-1L and FIGS. 2A-2C: The interaction formation was then divided into three sections: The B. subtills section (Bs) consisted of the entire B. subtilis biofilm, excluding the thick wrinkle that surrounded S. plymuthica. The wrinkle (direct contact area also designated interaction area was separated simply by lifting it (Int). The S. plymuthica section (Sp) consisted of the entire S. plymuthica colony excluding the cells that were attached to the wrinkle.

For follow up experiments, results were obtained by taking both colonies with no additional separation. As controls, colonies of B. subtilis and S. plymuthica were grown alone and also analyzed.

Each section/Colony preparate was harvested, inserted into 500 μl phosphate-buffered saline (PBS) and mildly sonicated (3×5 sec at 15%). The cell-solutions were then diluted, plated, and incubated at 30° C. overnight to allow formation of colonies. Cells were easily distinguished on LB medium due to differences in size and colony shape. During the calibration, the capacity to distinguish the bacterial strains was confirmed on differential biofilm medium. For B. subtilis biofilm mutants, confirmation of the identity ofthe colonies was performed by dual plating: on non-selective LB, and LB with the appropriate antibiotics.

Scanning Electron Microscopy (SEM). Two-day old interactions of B. subtilis WT or ΔpksX, with S. plymuthica, were fixed overnight, at 4° C., with 2% glutaraldehyde, 3% paraformaldehyde, 0.1 M Sodium Cacodylate (pH 7.4), and 5 mM CaCl₂. Samples were washed twice for 15 min with double distilled water (ddW) and dehydrated with a series of ethanol washes: 30%, 50%, 70%, 96% and 100%. Samples were then dried overnight, on filter paper (Whatman), at room temperature, mounted and stored in vacuum. The mounted samples were sputtered with gold-palladium before examination by Scanning Electron Microscopy (SEM) XL30 with Field Emission Gun (FEI).

Plant colonization assay. To test bacterial interaction over roots, seeds of the annual Eruca sativa (Brassicaceae), originated from natural population (Westberg et al 2013) were germinated on NITCH (Nitsch and Nitsch 1969) agar plates at 25° C. with a 8/16-h day/night photoperiod. One-week-old seedlings were transferred to solid MSgg-medium plates and bacteria were inoculated (as described above) on the consecutive next day at a distance of 0.8 cm from root and between the inoculants. Samples were incubated in growth chamber with 8/16-h day/night photoperiods at 23° C. and 30° C. for five and three days, respectively.

Root exudate extraction and hydrophobicity fractionation. Root exudate of E. sativa seedlings was extracted from NITSCH-agar growth medium two weeks from germination. After seedlings were removed, exudate was obtained by extracting NITSCH-agar with isopropanol (3:2) followed by vigorous vortex and incubating at room temperature (RT) for 10 min. Samples were centrifuged in 10,000 rpm for 10 min to separate the aqueous phase. Exudate samples were evaporated in a Speedvac concentrator (Eppendorf) and rehydrated in distilled water (dW). Prior to biofilms inoculation, plates were treated with either 50 μl drops of root exudate or NITSCH-agar (plant growth medium) extraction used as control.

pks expression analysis. pks expression in the presence of plant metabolites was analyzed as following: the luminescence intensity of cells harboring a P_{pksC}-lux transcriptional promoter fusion was analyzed. Cells were grown to a mid-logarithmic phase, diluted 1:100 in 150 μl liquid MSgg medium, either with or without root exudate. Cells were grown in a 96-well microplate (Thermo Scientific, Roskilde, Denmark) with agitation at 30° C. for 16 h in a microplate reader (Synergy 2, BioTek, Winooski, Vt., USA) measuring luminescence intensity and the optical density at 600 nm (OD₆₀₀) every 15 min.

Fluorescence microscopy. To validate PKS induction by specialized metabolites in a single cell resolution we grew cells harboring a pks-gfp transcriptional promoter fusion to a mid-logarithmic phase (grown in liquid LB) were diluted 1:100 in 150 μl liquid MSgg medium treated with 150 μM of Caffeic acid or Chlorogenic acid and incubated at 30° C. The culture was then placed on a carrying slide and covered with a poly L-lysin (SIGMA) treated coverslip. Cells were s visualized by fluorescence microscopy; Phase contrast and signal from Pₚₖₛ-gfp (green) are shown. Fluorescent cell count and intensity levels were recorded for 10 fields in each treatment, at least 1000 cells were quantified for each treatment.

ISR assay. E. sativa seedlings were grown on NITCH-agar for 10 days. Then each seedling was transferred to separate solid MSgg plate. In order to test the effects of either B. subtilis or S. plymuthica or their co-culture on the Induced systemic resistance (ISR), bacterial cells were inoculated aside to the root (as mentioned above) and incubated at 23° C. for five days. Alternatively and as indicated in the corresponding figure legend, we inoculated the bacterial cells directly on the root. Then the first leaf was infected by puncturing by needle and inoculating with the necrosis-causing pathogen, Pseudomonas syringae DC3000. Five days after infection with P. syringae, the area of black spots was measured to evaluate necrosis level.

Statistical methods. All studies were performed in duplicates or triplicates at least three separate and independent times. Data are expressed as average values±standard deviations of the means. For triple parties' interactions, data are expressed as average values±standard errors. Parametric testing was performed when appropriate after confirming that raw data were normally distributed. Data were analyzed by student's t test, used to determine if the set of treated versus the untreated control are different from each other (A paired t test comparing two sets of measurements) differ and P values of less than 0.1 were considered significant.

### Materials and Methods for Examples 6-12

Strains and Media All experiments were performed with B. subtilis NCBI3610 and its derivatives. Assays were carried out with the biofilm-inducing medium, MSgg: 5 mM potassium phosphate, 100 mM MOPS (pH 7), 2 mM MgCl₂, 50 μM MnCl₂, 1 μM ZnCl₂, 2 μM thiamine, 0.5% glycerol, 0.5% glutamate, 50 μg/ml tryptophan, 50 μg/mlphenylalanine 50 μg/ml threonine, 700 μM CaCl₂, and 50 μM (for growth assays) or 125 μM (for biofilm assays) FeCl₃¹. A solid MSgg medium was obtained by adding Bacto agar (Difco) to a final concentration of 1.5%. When necessary, selective medium was prepared with LB-agar or LB broth, supplemented with: 100 μg/ml ampicillin (amp) (AG Scientific), or 10 μg/ml kanamycin (kan) (AG Scientific), or 10 μg/ml chloramphenicol (cam) (Amresco), or 10 μg/ml tetracycline (tet) (Amresco), or 100 μg/ml spectinomycin (spec) (Tivan Biotech), or 1 μg/mlerythromycin (erm) (Amresco)+25 μg/ml lincomycin (linc) (Sigma Aldrich).

Plant colonization assay. To test the bacterial interaction over the plant roots, seeds of the annual Eruca sativa (Brassicacea), originated from natural population characterized by desert climate⁸ were germinated on NITCH⁹ agar plates at 25° C. with a 8/16-h day/night photoperiod. One-week-old seedlings were transferred to solid MSgg-medium plates and bacterial interactions were inoculated (as described above) on the consecutive day at a distance of 0.5 cm from root and 0.8 cm between the inoculants. The plates were incubated in growth chamber with a 8/16-h day/night photoperiod at 23° C. and 30° C. for five and three days, respectively.

Root exudate extraction and hydrophobicity fractionation. Root exudate of E. sativa seedlings was extracted from a solid NITCH-agar growth media two weeks from germination. After removing the seedlings from the growing plates, exudate was obtained by extracting growth agar with isopropanol (3:2) followed by vigorous vortex and incubating at room temperature (RT) for 10 min. Samples were centrifuged in 10,000 rpm for 10 min to separate the aqueous phase. Exudate samples were evaporated in a Speedvac concentrator (Eppendorf) and rehydrated in distilled water (DW). Exudate samples were then loaded on Sep-Pac C18 cartridge (Waters). Fractionation was performed using stepwise elution of 0%-100% methanol (DW, 10%, 20%, 30%, 40%, 60%, 80% and 100%). Again, to remove the methanol, fractions were evaporated in a Speedvac concentrator and rehydrated in DW before further examination of their effect.

ISR assay. Arabidopsis thaliana or Microtome tomato seedlings were grown on NITCH-agar for 10 days. Then each seedling was transferred to separate solid MSgg plate. In order to test the effects of either treated or untreated B. subtilis on the Induced systemic resistance (ISR), bacterial cells were inoculated aside to the root (as mentioned above) and incubated at 23° C. for five days. Alternatively and as indicated in the corresponding figure legend, we inoculated the bacterial cells directly on the root. Then the first leaf was infected by puncturing by needle and inoculating with the necrosis-causing pathogens, Pseudomonas syringae DC3000 or rhyzoctonia solani (Kuhn). Five days after infection with P. syringae, plants were further analyzed.

### Example 1

### B. Subtilis Engulfment of S. Plymuthica Cells

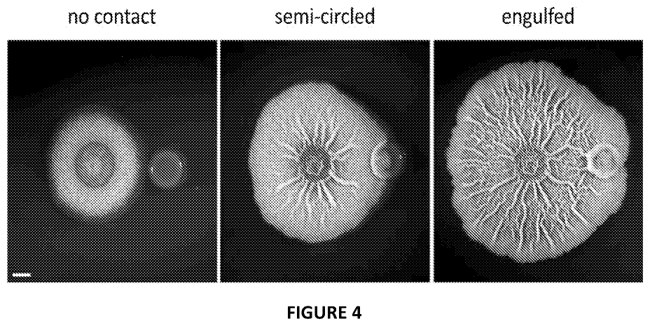

The Gram-positive B. subtilis bacterium can form a structured biofilm on a solid-air interface, where cells are encapsulated with a thick extracellular matrix. When grown undisturbed on a solid biofilm-inducing medium, B. subtilis biofilms form an almost perfect circular and symmetrical shape. To determine whether the B. subtilis biofilm development differs in the presence of a S. plymuthica colony, B. subtilis was inoculated on a solid biofilm medium at varying inoculation distances. By the third day of interaction, B. subtilis covered S. plymuthica with a thin, unstructured film that formed from the edges of the wrinkle to the center of the S. plymuthica colony, engulfing it completely (FIG. 4). Importantly, by the fourth day, the overlaying biofilm formed smaller wrinkles within the thick circular wrinkle and the B. subtilis colony continued to grow, over-passing S. plymuthica. The location and the shape of the center of the biofilm did not change during the interaction with S. plymuthica (FIG. 1A). In contrast to the near-perfect symmetry of the non-interacting B. subtilis biofilm, the interacting biofilm advanced asymmetrically towards the S. plymuthica colony, breaking from its circular shape (FIG. 1A). Surprisingly the WT strain expanded asymmetrically comparably to motility mutants and chemotaxis mutants towards the S. plymuthica colony (FIGS. 1B and 4).

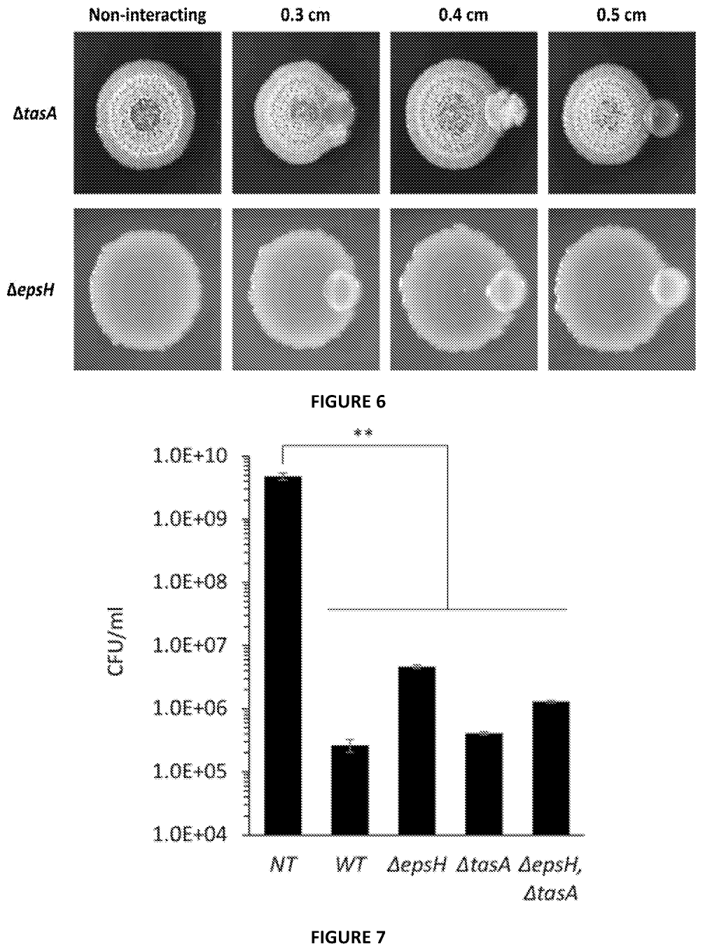

When grown solely, B. subtilis is capable of using the matrix components, primarily exopolysaccharides encoded by the eps operon, to slide towards new territories. Similarly, while both a tasA mutant, lacking the proteinous component of the matrix, and a mutant in eps had clear biofilm defects, only an eps mutant was incapable of expending asymmetrically towards its competitor (FIGS. 1C and 6).

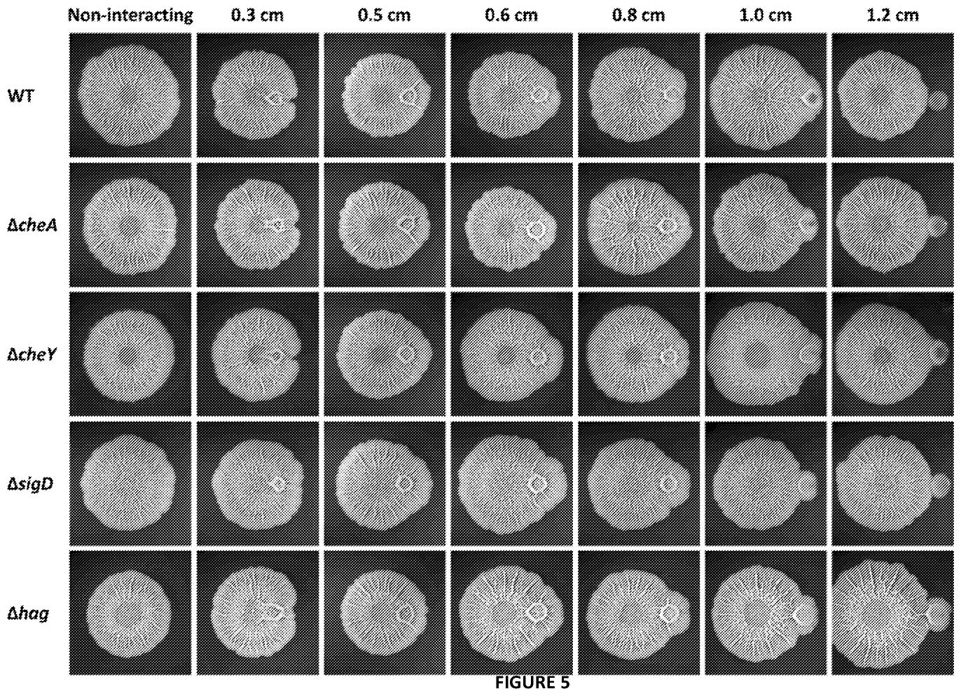

These results are supporting the hypothesis of the asymmetric expansion during interspecies interaction that is mediated by bacterial sliding motility (FIG. 1C). Intriguingly, dead S. plymuthica cells, but not the supernatant ofthe colony (FIG. 1D) robustly promoted asymmetrical expansion in a similar and even more pronounced manner than live S. plymuthica cells (FIG. 4). Both motility and chemotaxis B. subtilis mutants expanded assymetrically towards S. plymuthica colonies (FIG. 5). A ΔepsH strain, unable to produce the exopolysaccharides, could not expand towards S. plymuthica colonies (FIG. 6).

### Example 2

### PKS-Mediated Elimination of S. Plymuthica Cells

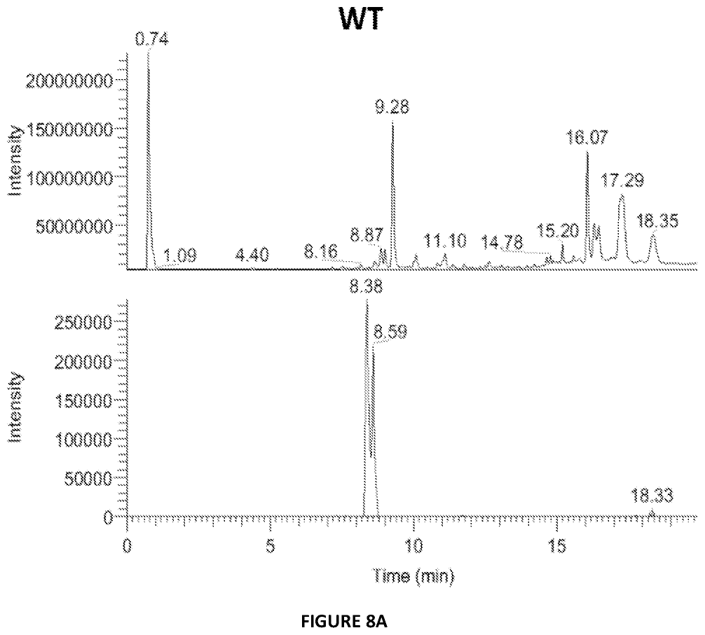

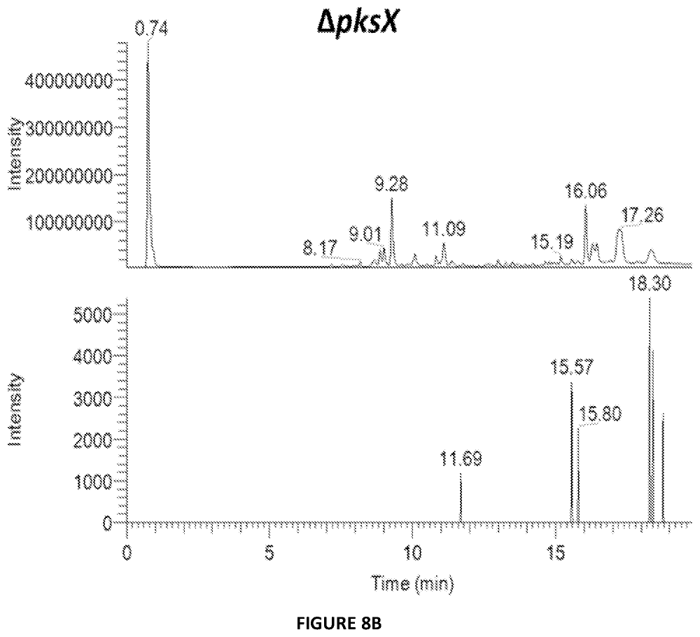

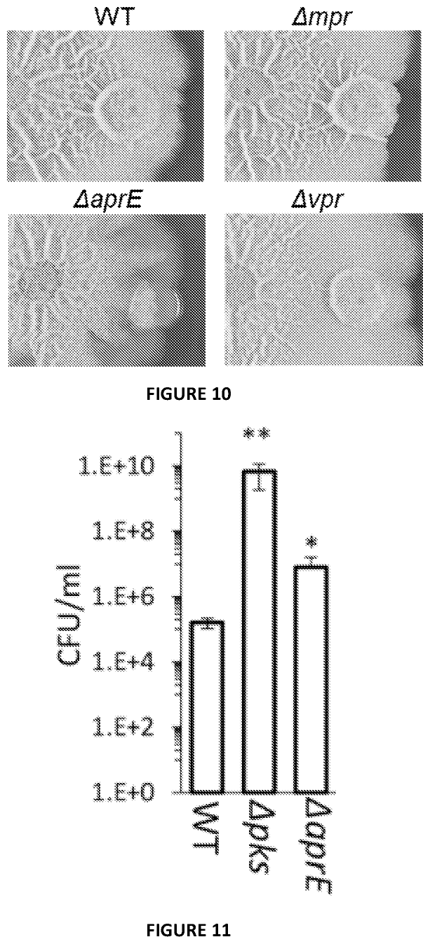

Following the engulfment, B. subtilis induced a dramatic reduction in the viability of S. plymuthica cells. However, although extracellular matrix is critical for the expansion, the matrix mutants were still able to eradicate the S. plymuthica colony (FIG. 7). Thus, engulfment promoted, but was not necessarily essential for, the eradication of S. plymuthica by B. subtilis. Two antibiotics, bacillaene and dihydrobacillaene are synthesized by enzymatic complex encoded in a pks gene operon, and were previously indicated to be involved in bacterial predator-prey interactions. We found that pks presence is essential to dramatically reduce the viability of the S. plymuthica colonies following interaction. Similar results were obtained using a ΔpksX, (FIG. 1E, left panel), and ΔpksA-Y (data not shown). In its presence (FIGS. 8A and 8B), B. subtilis are capable of lysing the S. plymuthica biofilms while having no impact on the engulfment of the competing colony (FIG. 1F). In the absence of the pks operon, S. plymuthica cells retained an intact morphology and a thick intercellular matrix. Once lyzed, S. plymuthica were further consumed by extracellular proteases (FIG. 10). pks was also found to be essential for B. subtilis competition with an additional beneficial biofilm former, P. chlororaphis (FIG. 11).

### Example 3

### PKS is Triggered by a Higher Order Interaction to Increase Plant Protection

While being highly effective at 30° C. reducing S. plymuthica cell counts by an order of magnitude, we found that a PKS-dependent killing is significantly much less efficient at 23° C. (FIG. 1E, right panel).

As we found that PKS products are essential for efficient prey-predator interaction between B. subtilis and its competitor, we evaluated whether their secretion can be induced under these conditions in the presence of the plant host. The effect of the plant host was especially potent, as it increased the killing of S. plymuthica cell-counts from two-fold to about 20-fold. In a mutant for polyketide synthesis, the host induced increased killing but on a significantly smaller scale (FIGS. 1G and 1H). The presence of the plant affected B. subtilis and S. plymuthica CFU when these bacteria where grown together. However, the presence of the plant had no effect on each of these strains when they were cultured alone (FIG. 11).

Next, B. subtilis effect on S. plymuthica growth was compared in an agar plate with or without root exudate. Root exudate enhanced B. subtilis killing of S. plymuthica (FIG. 1J). Root exudate had no effect on growth of B. subtilis (FIG. 1K), but affected pks activation (FIG. 1L).

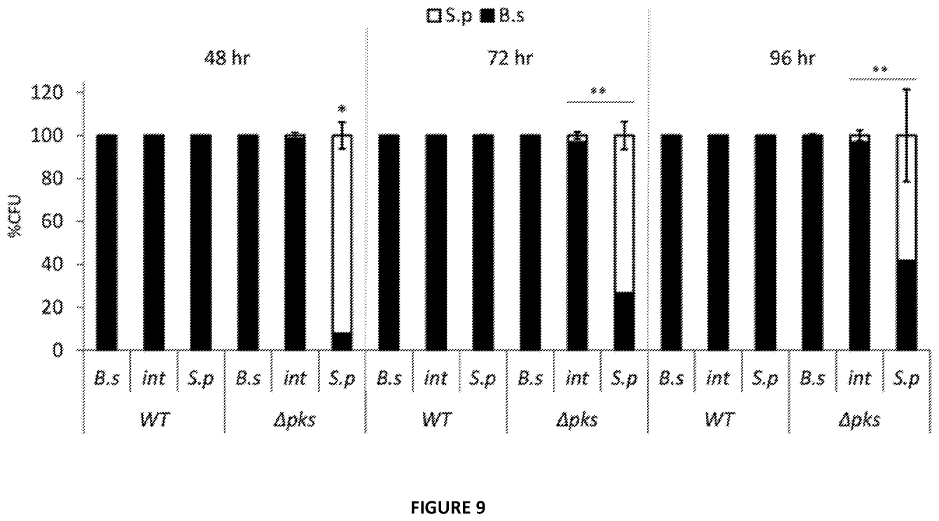

Next, CFU concentrations of B. subtilis and S. plymuthica inoculated on a solid biofilm medium was measured after 24, 48, and 72 hours. B. subtilis CFU concentration increased after 48 and 72 hours (FIG. 2A). S. plymuthica CFU concentration decreased after 48 and 72 hours (FIG. 2B). At 48 hours, the area in which B. subtilis and S. plymuthica interacted had only B. subtilis bacteria, and the S. plymuthic area had a high number of B. subtilis bacteria. After 72 hours, both the interaction area and the S. plymuthic area had almost solely B. subtilis bacteria (FIG. 2C). S. plymuthic killing by B. subtilis bacteria was heavily hampered in B. subtilis lacking the pks operon (FIG. 9).

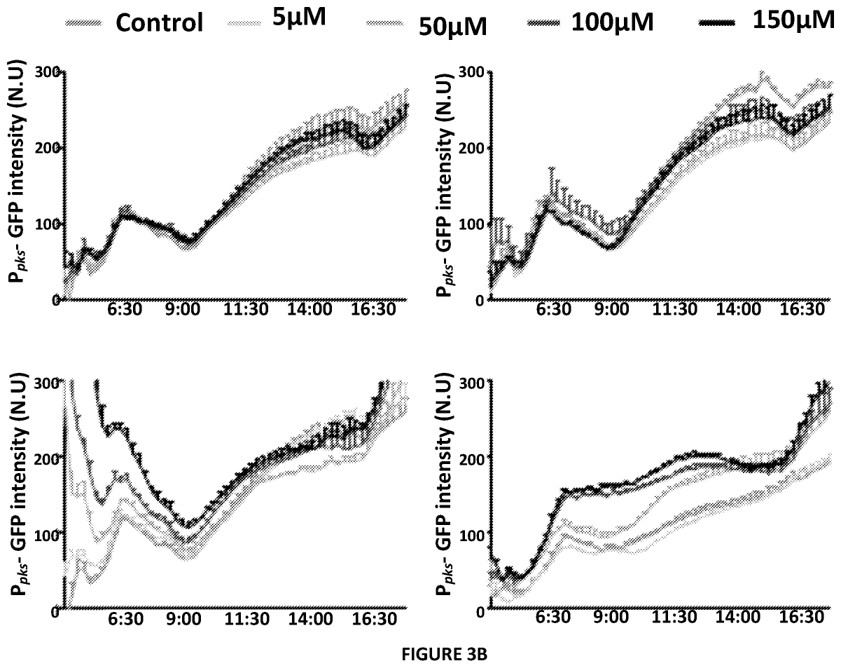

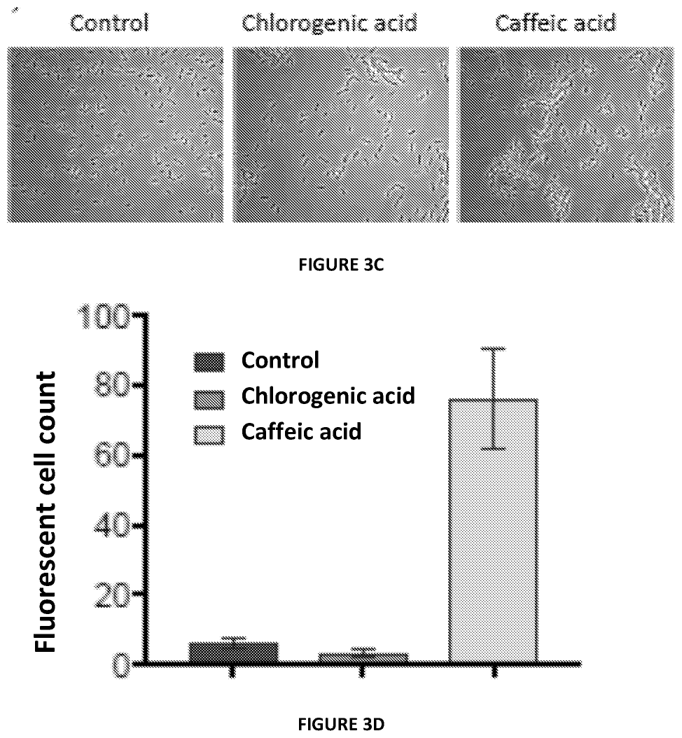

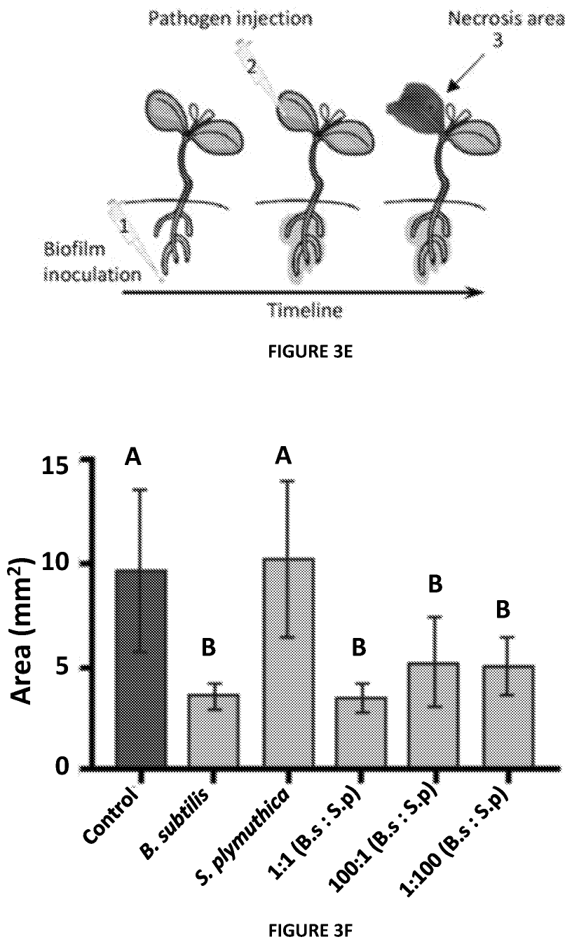

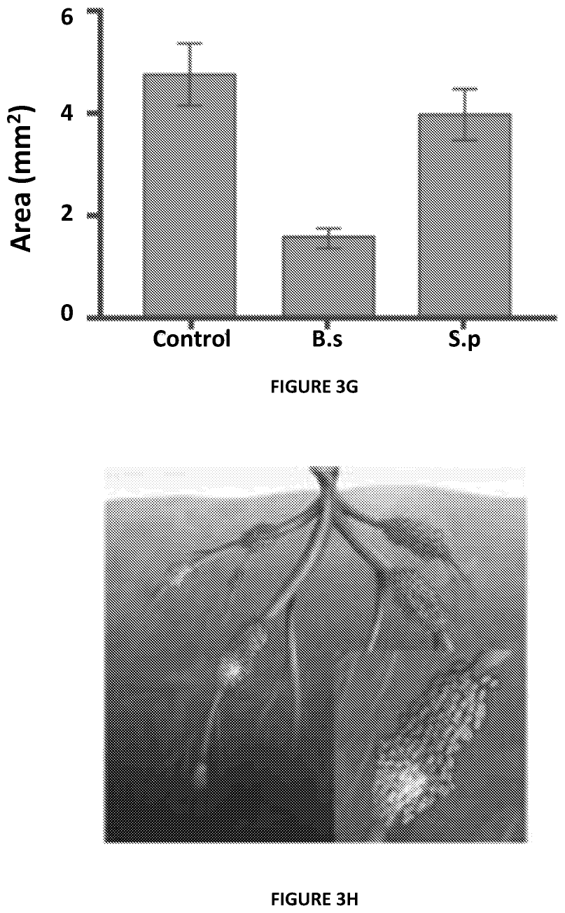

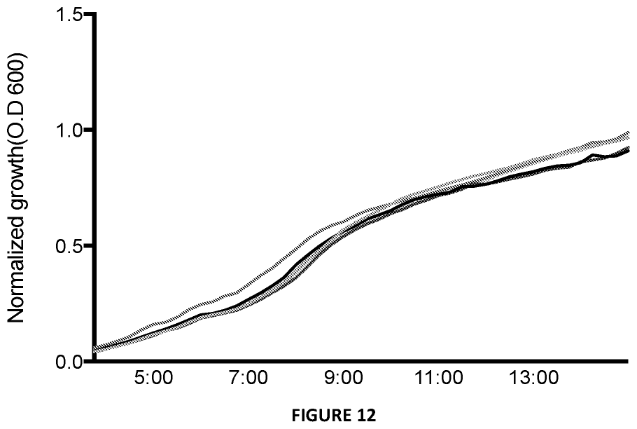

To test whether this enhanced killing is due to plant-bacteria interactions, we tested whether the secretions ofthe root are sufficient to induce the expression ofthe PKS operon. As shown, a purified fraction of the root extract was sufficient to significantly induce PKS expression (FIG. 3A). To identify the exact metabolite which is responsible for the induction we screened a library of over 250 natural products, putative positive heats were then purchased from Sigma and re-tested. Of the 250 metabolites, caffeic acid was found to be the only metabolite that significantly enhanced PKS expression (FIGS. 3B and 3C), indicating a highly specific mode of action. Caffeic acid induced the number of cells expressing the pks operon, while retaining the average expression level comparable to the control (FIGS. 3C and 3D), and with no impact on planktonic growth (FIG. 12), confirming it can act as a plant-derived signal.

### Example 4

### B. Subtilis Secreted Molecules Kill of S. Plymuthica Cells

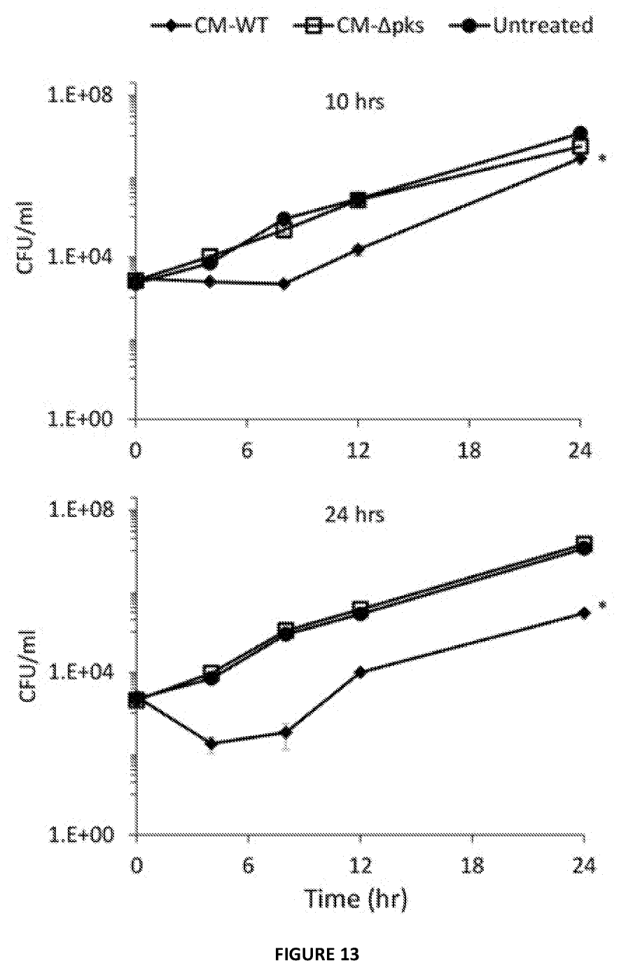

To further elucidate the mechanism by which B. subtilis kill S. plymuthica cells, B. subtilis supernatant was obtained and applied to S. plymuthica growing in a liquid biofilm medium. 50% [volume/volume] of B. subtilis WT supernatant collected at 10 hours and 24 hours reduced the concentration of S. plymuthica CFU. This effect was not seen in supernatant of B. subtilis Δpks cells (FIG. 13).

### Example 5

### Recruiting B. Subtilis Biofilms Increased the Plant Resistance to Plant Pathogens Compared with the Recruitment of S. Plymuthica Communities

A secondary metabolite produced by the plant induced antibiotic production of B. subtilis which provides an advantage to this bacterium over its competitors. Both B. subtilis and S. plymuthica are considered bio-control species, but their relative contribution to plant fitness was not known. In our studies, biofilms were established on E. sativa plants prior to their challenge with P. syringae (FIG. 3E). B. subtilis biofilms and B. subtilis-S. plymuthica mixtures, where B. subtilis overcomes S. plymuthica, provided a significantly enhanced systemic resistance to pathogens, as evaluated by the capacity of P. syringae to infect the leaves (FIGS. 3F and 3G).

Conclusion: It was shown previously that in a biofilm, cells use a variety of mechanisms to coordinate activity within the community, as well as across species. In many instances, biofilms provide beneficial effects to other organisms, e.g., bio-control agents form biofilms on the surface of plant roots, thereby preventing the growth of various pathogens, similarly to probiotic bacteria in the gut.

We show here that the plant host and B. subtilis biofilm can act as a “super-organism” to establish a beneficial community for the plant host (FIG. 3H). We focused on S. plymuthicam because B. subtilis and S. plymuthica simultaneously colonize plant roots, and thus are good candidates for studying interspecies interactions. It was shown previously that in pairwise species combinations, the great majority of interactions are overall negative. However, when two bacterial species and a eukaryote host are mixed together, the outcomes of the competition between these bacterial biofilms can be determined by the plant host. Surprisingly, host-dependent bacterial polyketide synthesis leads to the eradication of S. plymuthica by B. subtilis. This higher order interaction results in increased fitness to the plant host, as B. subtilis root-associated communities provide a significantly enhances systemic resistance versus pathogens.

Our finding provides a simple example of high order interactions that shape microbiomes: the host modulates antibiotic production in the desired bacterial colonizers, providing the colonizers a clear advantage over less attractive potential residents.

### Example 6

### Procurement of Root-Inhibitor of Dispersal (RID)

Under iron starvation, plant roots block the dispersal of beneficial bacterial biofilms. We used a novel method to identify and purify root-derived metabolites regulating beneficial biofilm stability and maintenance over the root. From a complex screen of tomato exudates containing the root secretome, a small molecule, termed Root Inhibitor of Dispersal (RID), was identified that blocks the disassembly of B. subtilis biofilms in vitro and in vivo under specific environmental conditions, such as iron starvation.

The active extract produced by plants starved for iron was run through HPLC chromatography, followed by Liquid chromatography-mass spectrometry (LC-MS). Importantly, our assay initially yielded non-conclusive results. While several small molecule candidates were suspected, none of them, when either purchased or synthesized enhanced the stability of biofilms of B. subtilis under laboratory conditions, or increased root coverage at late time points after the initial inoculum.

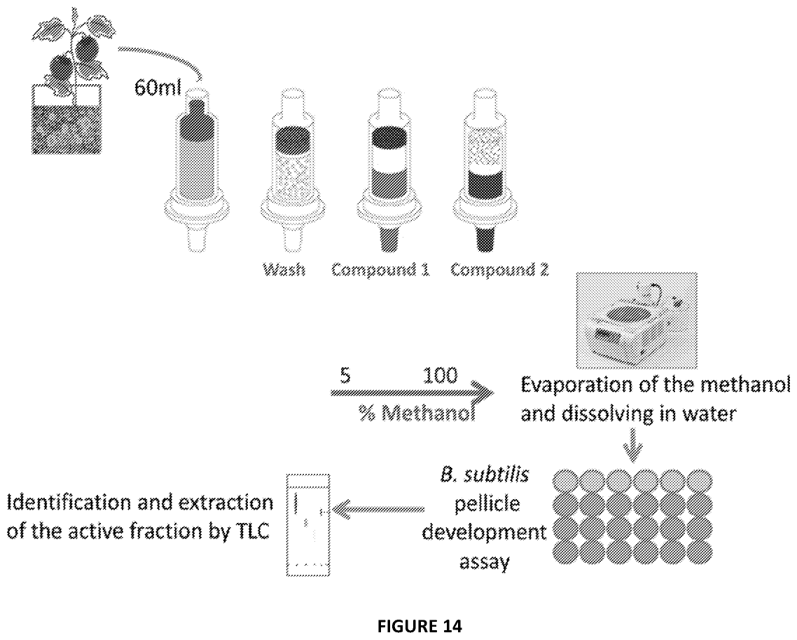

The procurement protocol was then modified based on preliminary mass spectrometry data to include a dansylation reaction. Dansyl groups are added to the potential amines, followed by thin layer chromatography (TLC; FIG. 14). Plants were grown under iron starvation over agrophonic beads. Exudates were collected, purified, analyzed for beneficial effects on the plant protector B. subtilis, after derivatization with dansyl chloride, and analyzed by TLC.

This combined protocol identified RID as caffeoyl putrescine, a naturally produced plant molecule containing two functional groups: caffeic acid (A) and the polyamine putrescine (B).

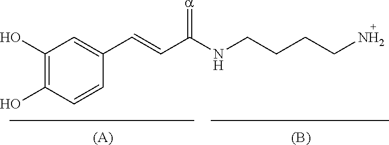

Both are soluble and commercially available (Sigma, Sigma-00625, Sigma-P7505).

### Example 7

### Caffeic Acid Alone or in Combination with Putrescine Improved Biofilm Stability

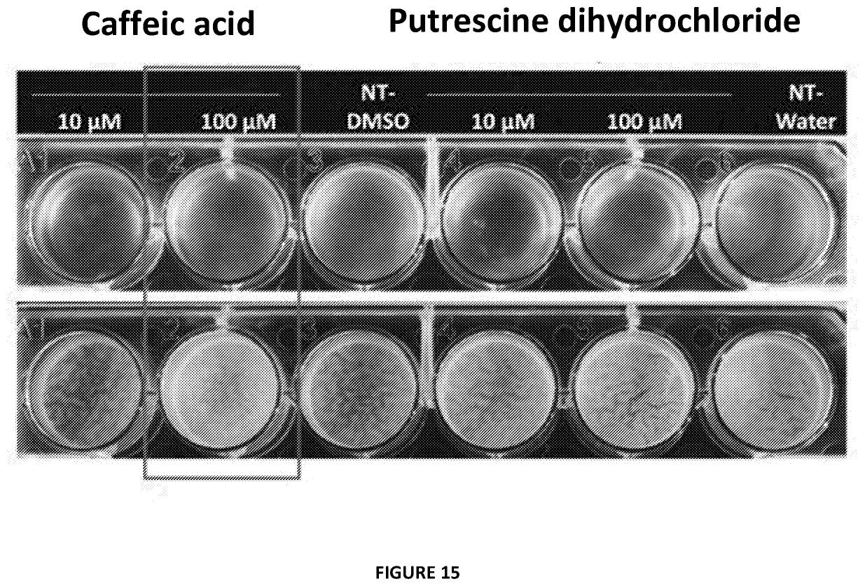

The characterization of the molecules on beneficial biofilms yielded several encouraging conclusions: first, while putrescine alone had little or no biological effect, the application of caffeic acid alone (Sigma) increased the stability of bacterial beneficial biofilms for up to 4 days under laboratory conditions at sub molar concentration (100 μM) (FIG. 15).

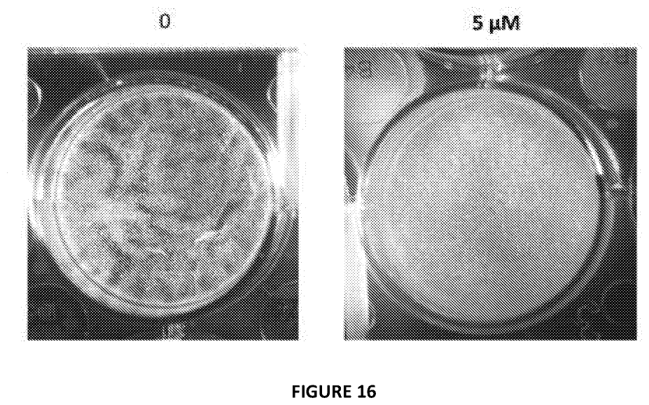

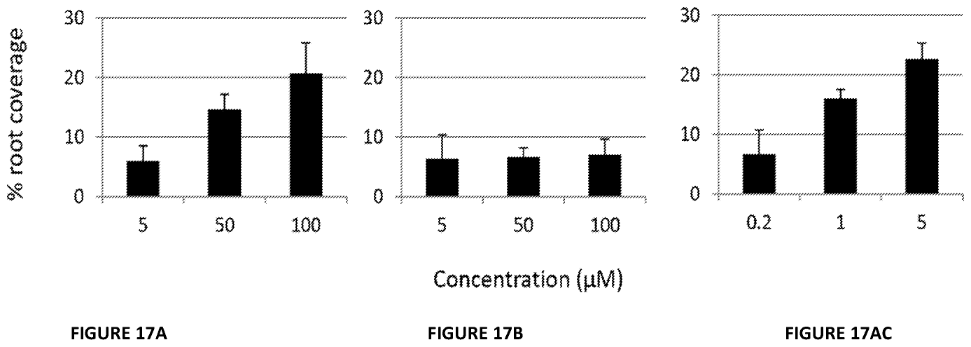

Importantly, a combination of 1-5 μM each of putrescence and caffeic acid also significantly prolonged the maintenance of beneficial biofilms in vitro, indicating that the two molecules work synergistically (FIG. 16-FIGS. 17A-17C).

Finally, caffeic acid alone or in combination with putrescine sustained protective biofilms in planta on tomato roots using a reproducible quantitative assay (FIGS. 17A-17C).

Thus, our system demonstrating plant bio-protection may be used to determine the minimal concentration of each active residue necessary for protection, the combinations of the different components of RID that provide protection, and the synergy between the components.

### Example 8

### RID Significantly Improved Bacillus Subtilis-Mediated Resistance of Plants to Pathogenic Infections

RID's effect on Bacillus control of multiple fungal and bacterial plant diseases was investigated. Using in vitro and in vivo (in soil) methodologies, the effect of RID-stabilized Bacillus communities on plant-associated biofilms that protect plants from fungal and bacterial pathogens was examined.

Bacillus strain B. subtilis 3610 (B.S) was maintained on LB plates. 1000 cells of B.S. with or without the addition of 5 μM of RID were added to tomato plants grown on agrophonic beads. Plants were incubated at 28±2° C. for 15 days.

Resistance of tomato plants to the bacterial pathogen pseudomonas syringae pv. Tomato after Bacillus subtilis exposure (with and without RID) were investigated.

P. syringae strain DC3000 pv tomato (P.S) was grown in LB at 37° C. to OD₆₀₀=0.2 to 0.3 (3*10⁸ cells per ml) and diluted to 10,000 cells retrospectively (w/v) into the plant media. Plants were further incubated at 28±2° C. for 10 days and the growth ofthe pathogen was measured and compared with the control. Three replications were maintained for each assay. SD represents the average of three independent experiments.

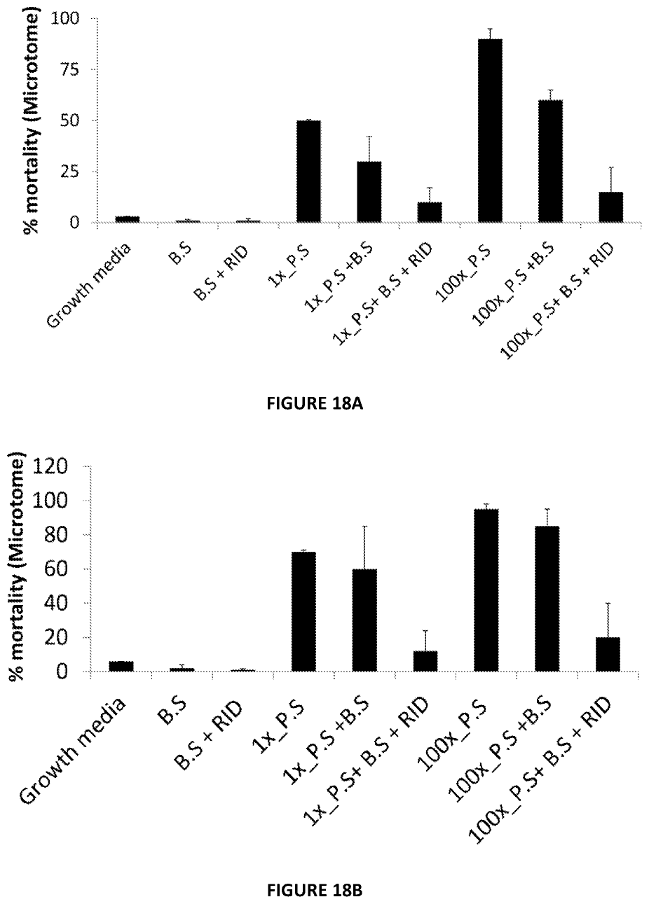

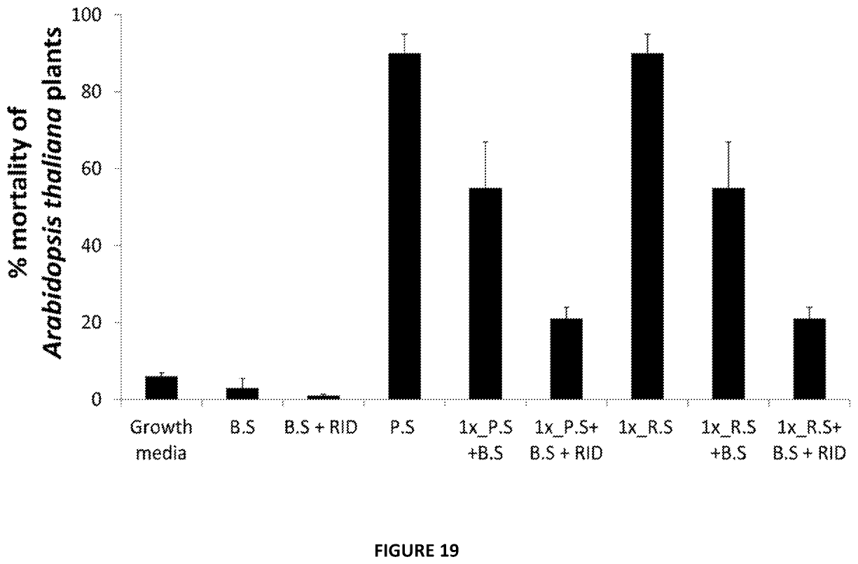

RID enhanced the protective effects of Bacillus subtilis on tomato plant mortality after rhyzoctonia solani (FIG. 19) and pseudomonas syringae pv. Tomato infections (FIGS. 18A, 18B and 19). RID's enhancement of Bacillus subtilis protection against pathogens was demonstrated after administration of both low and high doses of pathogen (FIGS. 18A, 18B and 19).

Next, the effect of RID on protection of Arabidopsis against pathogens was investigated. Arabidopsis represents the genus of the family Brassicaceae, a medium-sized and economically important family of flowering plants, designated unofficially as the mustards, mustard flowers, the crucifers, or the cabbage family. Resistance of Brassicaceae plants to the bacterial pathogen pseudomonas syringae and to the fungal pathogen rhyzoctonia solani was investigated.

Bacillus strain B. subtilis 3610 (B.S) was maintained on LB plates. 1000 cells with or without 5 μM of purified RID were added to Arabidopsis thaliana plants grown on MS solid media. Plants were incubated at 26±2° C. for 5 days. P. syringae (P.S) or R. solani (R.S) were grown in TSB at 37° C. to OD₆₀₀=0.2 to 0.3 (3*10⁸ cells per ml) and diluted to 100 cells, or 10,000 cells retrospectively (w/v) into the plant media. Plants were further incubated at 28±2° C. for 5 days and the growth of the pathogen was measured and compared with the control. Three replications were maintained for each assay.

Our results (FIG. 19) demonstrated that indeed purified RID can significantly improve the protection of Brassicaceae plants afforded by Bacillus subtilis from pseudomonas syringae (a bacterial pathogen related to pseudomonas syringae vs. tomato) and rhyzoctonia solani (a fungal pathogen toxic to Brassicaceae).

### Example 9

### Caffeic Acid Alone or in Combination with Putrescine Improves Bacillus Subtilis-Mediated Resistance of Plants to Pathogenic Infections

Bacillus strain B. subtilis 3610 (B.S) are maintained on LB plates. 1000 cells of B.S. with or without the addition of 5 μM of caffeic acid or 5 μM of putrescine, or 2.5 μM each of caffeic acid and putrescine are added to tomato plants grown on agrophonic beads. Plants are incubated at 28±2° C. for 15 days.

Bacillus subtilis alone protects tomato plants from pseudomonas syringae pv. Tomato infection. Application of caffeic acid (Sigma) alone or in combination with putrescine increased tomato plant survival after pseudomonas syringae pv. Tomato infection. The combination of caffeic acid and Bacillus subtilis and the combination of caffeic acid, putrescine, and Bacillus subtilis are most effective at increasing tomato plant survival after pathogenic infection. However, the application of putrescine alone has little or no biological effect on tomato plant survival 28 days after pseudomonas syringae infection.

### Example 10

### Polyamines, Amines and Analogues or Derivatives of Caffeic Acid and Putrescine

Certain polyamines, amines, analogues or derivatives of caffeic acid and putrescine, as described hereinabove, are expected to demonstrate a protective effect on a broad range of plant species from infection. Polyamines, amines, analogues or derivatives that afford better protection than caffeic acid and caffeic acid combined with putrescine, and especially those that have an effect at nanomolar concentrations will be selected.

### Example 11

### Caffeic Acid-Putrescine Conjugates

Caffeic acid-putrescine conjugates are expected to demonstrate a protective effect on a broad range of plant species from infection with a broad range of microbial and fungal pathogens.

### Example 12

### RID and Its Related Products Also Protect Wheat, Potato, and Corn Plant Roots from Infection with Xanthomonas Campestris and Fusarium Graminearum

Caffeic acid, putrescine, analogues or derivatives of caffeic acid and putrescine, or their conjugates protect wheat, potato and commercial com plant roots from infection with a broad range of bacterial species, including Xanthomonas campestris. Xanthomonas campestris is the bacterium that causes Black Rot, which is the main disease of cruciferous plants. Caffeic acid, putrescine, analogues or derivatives of caffeic acid and putrescine, or their conjugates protect wheat, potato and commercial corn plant roots from infection with a broad range of fungal species, including Fusarium graminearum. Fusarium graminearum causes Fusarium head blight, which is a devastating disease that leads to extensive yield and quality loss of wheat and barley crops. Losses in years of outbreak are in the billion dollar range.

### Example 13

### Caffeic Acid Alone or in Combination With Putrescine Increases the Efficiency of Extracellular Matrix Production in B. Subtilis and Increases the Synthesis of Anti-Bacterial and Anti-Fungal Molecules in B. Subtilis

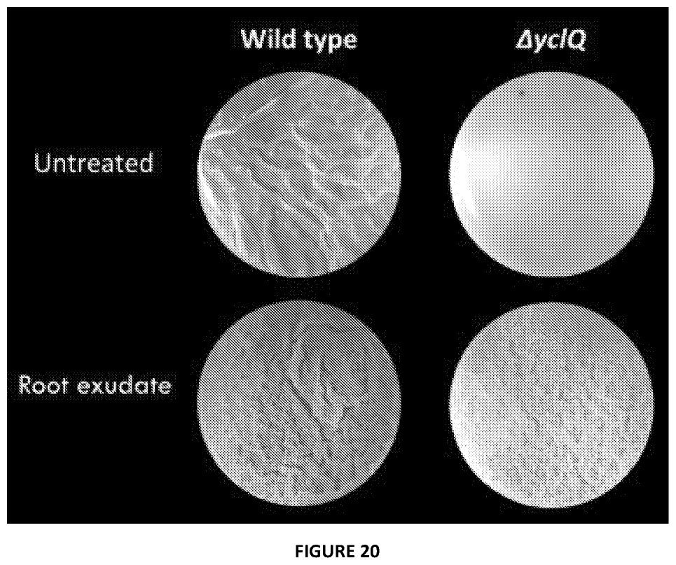

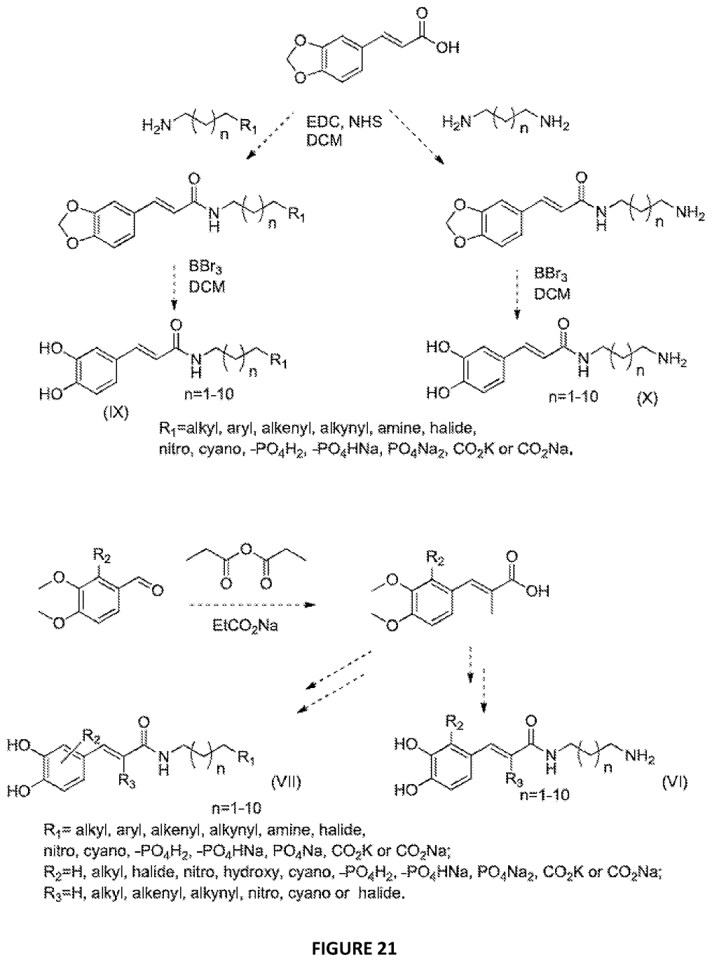

Purified RID compensated for the biofilm defect of a B. subtilis strain defective in iron uptake (FIG. 20). Caffeic acid binds iron tightly, and putrescine generates pores in the bacterial cell wall. Thus, the possibility that the synergy between RID's components is an outcome of the intracellular capacity of the bacteria to recycle ferric iron bound to caffeic acid, after putrescine promotes its entry to the cell is investigated. RID promotes iron uptake by specifically binding to iron-uptake systems of B. subtilis.

We will confirm the effect of single molecule, conjugate caffeoyl putrescine as well as mixtures on the efficiency of extracellular matrix production, anti-bacterial molecule synthesis and anti-fungal molecule synthesis. We will also explore and determine the contribution of each active molecule/molecule combination to the inhibition dispersal-related genes.

Identification of RID and its components, and establishment of its in vitro and in vivo (in planta) activity, has generated knowledge that may be used for developing ‘protective armor’ for plants against bacterial and fungal pathogens.

### Example 14

### Regulation and Function of Δpks Operon

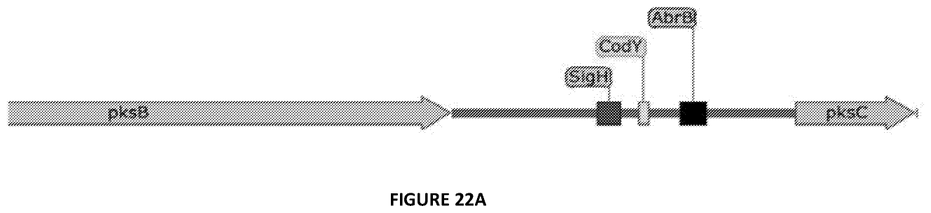

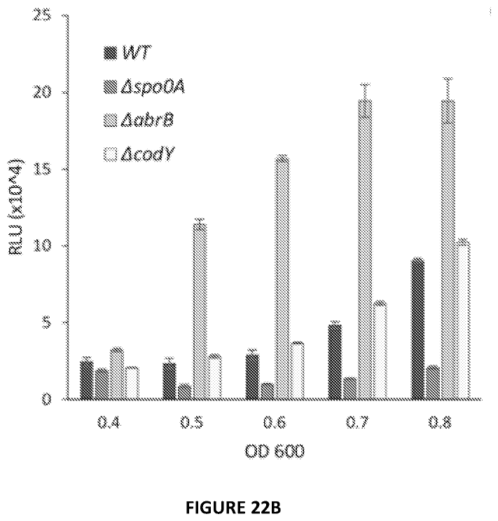

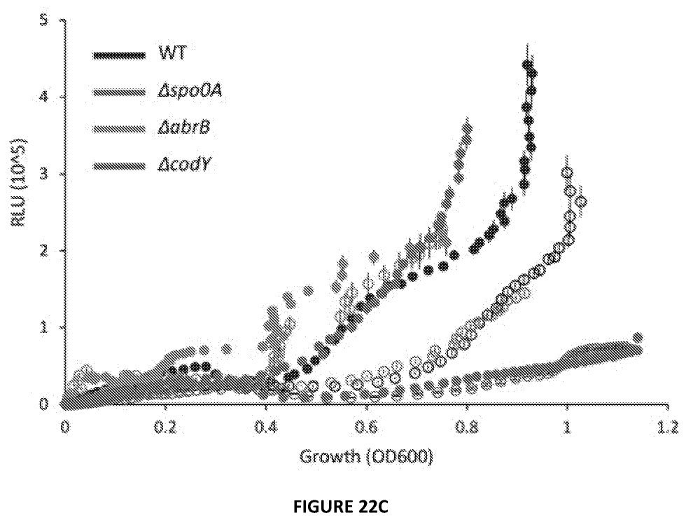

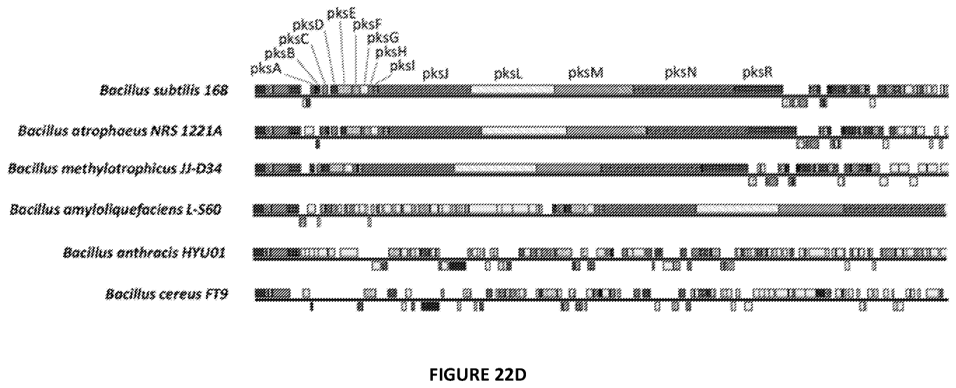

To gain further understanding on the role of the Δpks operon (FIG. 22A) on B. subtilis effect, a numer ofB. subtilis strains carrying a P_{pksC}-lux construct were created. These strains showed showed different planktonic growth (FIG. 22B). Root exudate increased PKS activation (FIG. 22C). FIG. 22C further shows how different mutations affected PKS activation, as well as root exudate effect on PKS activation.

The genome of different Bacilus species was compared. Bacillus subtilis 168, Bacillus atrophaeus NRS 1221A, Bacillus amyloliquefaciens L-S60 and Bacillus methylotrophicus JJ-D34 revealed a conserved PKS operon (pksA-R). In contrast, Bacillus anthracis HYU01 and Bacillus cereus FT9 strains lacked the pks operon altogether (FIG. 22D).

### Example 15

### An Ester of Caffeic Acid Induces Antibiotic Production

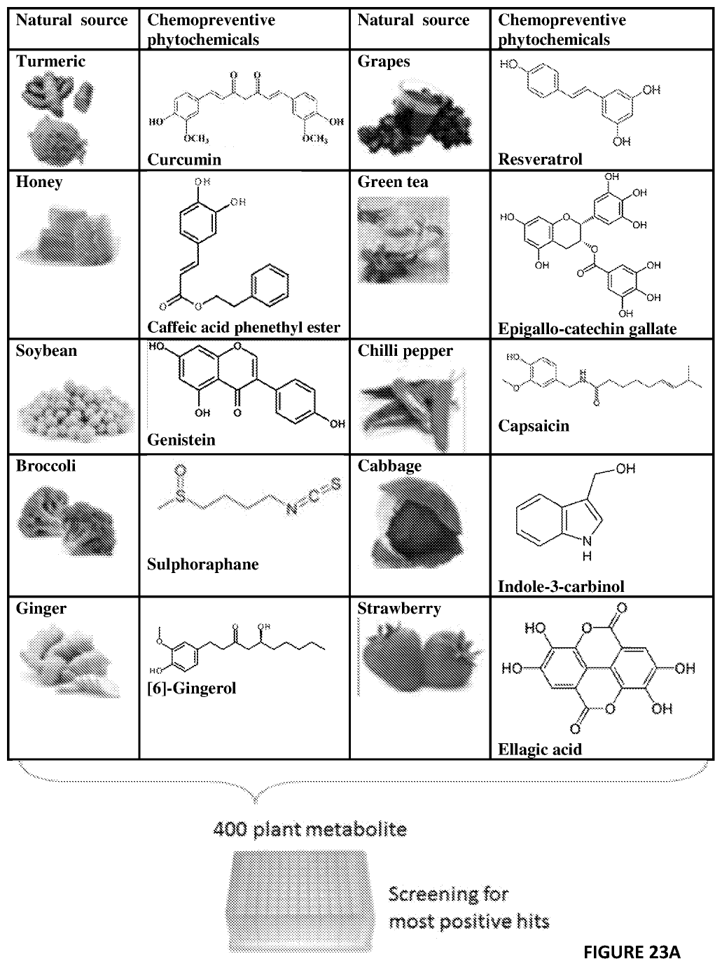

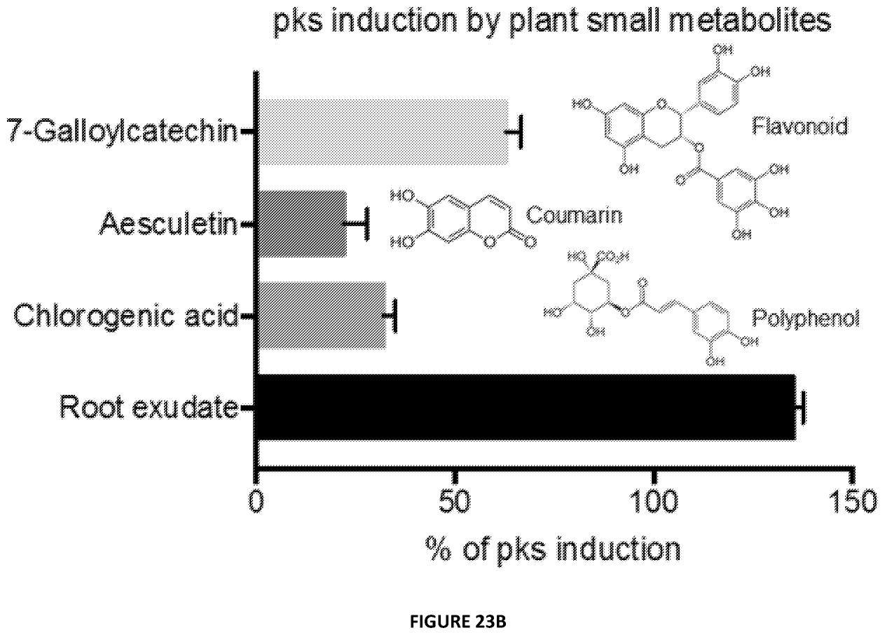

A library of 400 plant metabolites was screened for pks induction using B. subtilis cells carrying the P_{pksC}-lux construct (FIG. 23A). The molecules 7-galloylcatechin, aesculetin, and chlorogenic acid induced pks activation (FIG. 23B), suggesting that these molecules induce antibiotic production in B subtilis cells.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of synthesizing an anti-microbial agent in bacteria, enhancing the production of an anti-microbial agent in bacteria, activating the pks gene operon in bacteria having a pks gene operon, or a combination thereof, the method comprising administering to said bacteria a composition comprising a hydroxycinnamic acid, or a conjugate, analogue or derivative thereof.

2. The method of claim 1, wherein said anti-microbial agent comprises an anti-bacterial agent or an anti-fungal agent.

3. The method of claim 2, wherein said anti-bacterial agent comprises a polyketide antibiotic.

4. The method of claim 3, wherein said polyketide antibiotic comprises bacillaene, dihydrobacillaene, or a combination thereof.

5. (canceled)

6. A method for enhancing extracellular matrix production in bacteria, the method comprising administering to said bacteria a composition comprising a hydroxycinnamic acid, or a conjugate, analogue or derivative thereof.

7. The method of claim 1, wherein said bacteria are B. subtilis.

8. The method of claim 1, wherein said bacteria are in a biofilm, in a bacterial culture or on a root of a plant.

9. (canceled)

10. (canceled)

11. The method of claim 1, wherein the composition is indirectly administered to said bacteria.

12. The method of claim 11, wherein said bacteria are present on an organ of a plant and said composition is administered to said plant.

13. A method for inducing, enhancing, or maintaining biofilm formation by a beneficial rhizobacteria on a plant or a part thereof, the method comprising the step of administering to the plant an effective amount of a composition comprising a hydroxycinnamic acid, or a conjugate, analogue or derivative thereof.

14. The method of claim 13, wherein said biofilm comprises a B. subtilis biofilm.

15. A method for increasing resistance to disease in a plant, treating a disease in a plant, or suppressing the growth or survival of a biological agent on or near a plant, the method comprising administering to the plant a composition comprising a hydroxycinnamic acid, or an analogue or derivative thereof.

16. (canceled)

17. The method of claim 15, wherein said disease is characterized by an infestation or infection by a plant pathogen.

18. (canceled)

19. The method of claim 15, wherein said biological agent comprises a plant pathogen or a non-pathogenic bacterial species.

20. The method of claim 19, wherein said plant pathogen comprises a fungal pathogen or a bacterial pathogen.

21. The method of claim 20, wherein said fungal pathogen comprises Rhizoctonia solani or Fusarium graminearm.

22. (canceled)

23. (canceled)

24. The method of claim 20, wherein said bacterial pathogen comprises Pseudomonas syringae or Xanthomonas campestris.

25. (canceled)

26. (canceled)

27. The method of claim 19, wherein said non-pathogenic bacterial species comprises S. plymuthica, P. chlororaphis, or both.

28. The method of claim 13, wherein said plant comprises a crop plant.

29. The method of claim 28, wherein said crop plant comprises a Brassicaceae family plant.

30. The method of claim 29, wherein said Brassicaceae family plant comprises Brassica oleracea (cabbage, broccoli, Brussels sprouts, kohlrabi, or cauliflower), Brassica rapa (turnip), Brassica napes (rapeseed), mustard, Raphanus raphanistrum (radish), Armoracia rusticana (horseradish), Lepidium sativum (cress), Eutrema japonicum (wasabi), Eruca sativa (arugula), Nasturtium officinale (watercress), Brassica juncea, (brown mustard), or canola.

31. The method of claim 28, wherein said crop plant comprises a Solarium lycopersicum (tomato), Solanum tuberosum (potato), Zea mays (corn), or Tritieum aestivum (wheat).

32. The method of claim 13, wherein said composition is administered to said plant by soil injection, soil drenching, injection of the composition into plant vasculature, administered to the root of the plant, or a combination thereof.

33. (canceled)

34. The method of claim 1, wherein said hydroxycinnamic acid, or a conjugate, analogue or derivative thereof is selected from a group comprising caffeic acid, 7-galloylcatechin, aesculetin, and chlorogenic acid.

35. The method of claim 1, wherein said composition comprising hydroxycinnamic acid further comprises a polyamine.

36. The method of claim 1, wherein said hydroxycinnamic acid derivative comprises an amide derivative comprising a polyamine.

37. The method of claim 35, wherein said polyamine comprises putrescine.

38. The method of claim 36, wherein said hydroxycinnamic acid amide derivative comprises caffeoyl putrescine.

39. A method for isolating caffeoyl putrescine from the root or root exudate of a plant, the method comprising:
a. obtaining root exudate from said plant;
b. fractionating and purifying said exudate;
c. dansylating amine groups in the fraction of said exudate that blocks B. subtilis biofilm dispersal; and
d. resolving dansylated compounds from selected fractions using thin layer chromatography,
thereby isolating caffeoyl putrescine from the root or root exudate of said plant.

40. The method of claim 39, wherein fractionation of step (b) comprises HPLC chromatography followed by liquid chromatography-mass spectrometry (LC-MS).

41. The method of claim 39. wherein the fraction blocking B. subtilis biofilm dispersal is tested in step (c) with a pellicle development assay.

42. The method of claim 39, wherein the dansylation in step (c) comprises derivatization of amine groups with dansyl chloride.

43. The method of claim 39, wherein said plant was optionally grown under iron-deficient conditions.

44. A composition comprising a mixture of a) a hydroxycinnamic acid or an analogue or derivative of hydroxycinnamic acid and b) a polyamine or an analogue or derivative of a polyamine.

45. The composition of claim 44, wherein said hydroxycinnamic acid or an analogue or derivative of hydroxycinnamic acid comprising caffeic acid, 7-galloylcatechin, aesculetin, and chlorogenic acid.

46. The composition of claim 45, wherein said composition comprises caffeic acid, 7-galloylcatechin, aesculetin, or chlorogenic acid at a concentration of 1-150 μM.

47. The composition of claim 45, wherein said composition comprises caffeic acid, 7-galloylcatechin, aesculetin, or chlorogenic acid at a concentration of 1-5 μM.

48. The composition of claim 44, wherein said polyamine comprises putrescine.

49. The composition of claim 48, wherein said composition comprises caffeic acid, 7-galloylcatechin, aesculetin, or chlorogenic acid, and putrescine at a concentration of 1-5 μM each.

50. The composition of claim 44, wherein said composition further comprises a botanically compatible vehicle.

51. The method of claim 6, wherein said bacteria are B. subtilis.

52. The method of claim 6, wherein said bacteria are in a biofilm, in a bacterial culture or on a root of a plant.

53. The method of claim 15, wherein said plant comprises a crop plant.

54. The method of claim 15, wherein said composition is administered to said plant by soil injection, soil drenching, injection of the composition into plant vasculature, administered to the root of the plant, or a combination thereof.

55. The method of claim 6, wherein said hydroxycinnamic acid, or a conjugate, analogue or derivative thereof is selected from a group comprising caffeic acid, 7-galloylcatechin, aesculetin, and chlorogenic acid.

56. The method of claim 13, wherein said hydroxycinnamic acid, or a conjugate, analogue or derivative thereof is selected from a group comprising caffeic acid, 7-galloylcatechin, aesculetin, and chlorogenic acid.

57. The method of claim 15, wherein said hydroxycinnamic acid, or a conjugate, analogue or derivative thereof is selected from a group comprising caffeic acid, 7-galloylcatechin, aesculetin, and chlorogenic acid.

58. The method of claim 39, wherein said hydroxycinnamic acid, or a conjugate, analogue or derivative thereof is selected from a group comprising caffeic acid, 7-galloylcatechin, aesculetin, and chlorogenic acid.

59. The method of claim 44, wherein said hydroxycinnamic acid, or a conjugate, analogue or derivative thereof is selected from a group comprising caffeic acid, 7-galloylcatechin, aesculetin, and chlorogenic acid.


SEQUENCE LISTING (6 sequences)

SEQ ID NO: 1 - DNA, 57 bp, Artificial Sequence
  Primer 5′ Ppks-lux P1
gtcctagtaaggtcgacaggaggactctctgcaaatcgcccggccattcgataaagg

SEQ ID NO: 2 - DNA, 57 bp, Artificial Sequence
  Primer 3′ PpksC-lux P2
gtatgtaagcaaaaagtttccaaatttcattctctcaaagccacccttccgattagt

SEQ ID NO: 3 - DNA, 26 bp, Artificial Sequence
  Primer P1
tcgatatggatgaagtcggccaggaa

SEQ ID NO: 4 - DNA, 49 bp, Artificial Sequence
  Primer P2
caattcgccctatagtgagtcgtccgcagcttgcagcatggagttaata

SEQ ID NO: 5 - DNA, 55 bp, Artificial Sequence
  Primer P3
ccagcttttgttccctttagtgagtcaggcttatatcaaggcgagaaatgtagtt

SEQ ID NO: 6 - DNA, 26 bp, Artificial Sequence
  Primer P4
ttctgtaaggcacccactctccattc